(19) United States
(12) Patent Application Publication
(10) Pub. No.: US 2014/0045706 A1
(43) Pub. Date: Feb. 13, 2014

(54) METHODS AND SYSTEMS FOR HAPLOTYPE DETERMINATION

(71) Applicants: Jian-Bing Fan, San Diego, CA (US); Jeffrey S. Fisher, San Diego, CA (US); Fiona Kaper, San Diego, CA (US)

(72) Inventors: Jian-Bing Fan, San Diego, CA (US); Jeffrey S. Fisher, San Diego, CA (US); Fiona Kaper, San Diego, CA (US)

(73) Assignee: ILLUMINA, INC., San Diego, CA (US)

(21) Appl. No.: 13/980,009

(22) PCT Filed: Feb. 24, 2012
(86) PCT No.: PCT/US12/26623

§ 371 Date: Sep. 23, 2013

(30) Foreign Application Priority Data
Feb. 25, 2011 (US) 61446890
Jul. 20, 2011 (US) 61509960

(51) Int. Cl. C12Q 1/68

(52) U.S. Cl. CPC C12Q 1/6809
USPC 506/2; 435/6.11; 506/17

(57) ABSTRACT

Embodiments of the present disclosure provide methods and systems for determining the haplotype of a biological sample. Particular embodiments provide methods for long range haplotyping of a genome.

This application claims priority to U.S. provisional patent application Ser. No. 61/446,890 filed Feb. 25, 2011 and U.S. provisional patent application Ser. No. 61/509,960 filed Jun. 20, 2011, both of which are incorporated herein by reference in their entireties.

### BACKGROUND

The efforts of the Human Genome Project opened a broader window to the human genetic code. The work to further unlock the human genome is ongoing, for example using high-throughput sequencing technologies. The HapMap (Haplotype Map) Project is a global scientific effort directed at discovering genetic variants that lead to disease by comparing genomic information from people without a particular disease to those with that disease. Alleles, one or more forms of a DNA sequence for a particular gene, can contain one or more different genetic variants and identifying haplotypes, or combinations of alleles at different locations, or loci, on a particular chromosome is a main focus of the HapMap Project. Identified haplotypes where the two groups differ might correlate to locations of genetic anomalies that cause disease. As such, HapMap results will help to describe the common patterns of genetic variation in humans and whether those variations are potentially correlated to disease.

The information gained from these efforts, even though the sequences are incomplete and there are gaps and sometimes mistakes, provides a valuable tool in helping to decipher the genetics behind diseases and disorders. Unfortunately, the cost in performing such large scale sequencing is still very high and the technologies to provide more in depth information, such as single chromosome haplotyping, phasing of alleles or target sequences, are illusive. What are needed are additional tools and technologies to unlock more information from the human genome.

### SUMMARY

Current genotyping technologies can provide an investigator with a subject's genetic makeup. However, the technology is limited with regard to providing a convenient and scalable means to determine what sequences are adjacent, or proximal, to one another on one chromosome as opposed to those adjacent, or proximal, on the other chromosome. FIG. 2 exemplifies a dilemma where a genotype of a subject can be determined, however insufficient information is available to determine whether a sequence of interest (e.g., allele, single nucleotide polymorphism (SNP), copy number variant (CNV), genetic insertion or deletion (indel), etc.) is located on the same chromosome as another sequence of interest. For example, for a mixed population of chromosomes in a sample from a subject (FIG. 2A) it may be possible to determine an exemplary genotype from data (FIG. 2B). However, insufficient information is provided to determine how heterozygous alleles are grouped together on a chromosome (haplotyping). For example, it is unknown whether Parent A (Pₐ) is providing alleles α and γ, Parent B (P_{b}) alleles α′ and γ′ (FIG. 2C), or whether they are mixed (FIG. 2D). It is even more difficult to determine which sequences of interest are present on the same chromosome when those sequences are located far apart, or distal or long-range to each other on a chromosome, thereby determining a chromosome's long-range haplotype or allelic phasing.

Embodiments of the present disclosure provide novel solutions for determining phased alleles regardless of their location with respect to each other on the chromosome (e.g., proximal or distal). During experimentation directed at solving current haplotyping challenges, it was discovered that providing an unbalanced or asymmetric distribution of genetic material provided a novel solution to the problem of accurate haplotyping of a subject. Optional amplification of target sequences following unbalanced distribution was particularly useful. The present invention is not limited to a particular mechanism. Indeed, an understanding of the mechanism is not necessary to practice the present invention. Nonetheless, it is contemplated that amplification signal intensity determines the haplotype of a chromosome based in part on differential amplification of the unbalanced material. For example, the ratio of different allelic signals determines which are found on a single chromosome thereby determining a phased haplotype for a sample. FIG. 3 exemplifies such an embodiment. An imbalance in the original sample distribution (as seen in 3B and 3D) is exploited and differential amplification demonstrates that α allele is phased, or grouped, with γ′ allele of Pₐ and that α′ is phased with γ on P_{b} (3E). Further, embodiments are not limited to a haploid sample but instead are effective when either a diploid sample (e.g., paired chromosomes, DNA inserts, YACs, BACs, cosmids, fosmids, etc.) or a haploid sample (e.g., genetic complement from a sperm, egg, complete hydatiform mole, etc.) is utilized.

The information gained from the phasing of alleles in a genome as provided by practicing the methods described herein finds utility in general research and discovery efforts as well as, for example, disease detection, therapeutics and higher confidence in HLA compatibilities for decreasing transplant rejection. For example, known haplotypes can be correlated to drug metabolism, drug discovery, disease states, cancers, disorders, transplant rejection risk and personalized healthcare initiatives to name a very few. Indeed, with regards to personalized healthcare once a subject's personal haplotype is known then the subject's specific disease correlation and therapeutic options can be designed specifically to meet the needs of that subject.

One embodiment of the present disclosure comprises methods for determining the haplotype of a nucleic acid sample by providing a fraction of a sample comprising a detectable imbalance between two or more sequences of interest in the nucleic acid sample and determining the haplotype of the nucleic acid sample based on said detectable imbalance. In some embodiments, a nucleic acid sample is from a genome or fragments thereof, wherein said genome is derived from one or more cells, for example approximately 1-100 cells. In some embodiments, the nucleic acid sample is from a mammal, preferably a human. In other embodiments, the nucleic acid sample is from a non-human mammal, a plant or a virus. In some embodiments, the nucleic acid sample comprises wild-type sequences at a sequence of interest whereas in other embodiments the nucleic acid sample comprises variant sequences at a sequence of interest. In some embodiments, the sequences of interest comprise a wild-type sequence at one sequence of interest and a variant sequence at another sequence of interest, or combinations thereof. In some embodiments, the variant sequences are selected from a group comprising single nucleotide polymorphisms, copy number variants, genomic insertions and genomic deletions. In some embodiments, a detectable imbalance between two or more sequences of interest in a sample is determined by fluorescence. In some embodiments, a detectable imbalance between two or more sequences of interest in a sample is determined by a nucleic acid sequencing technique, by a genotyping technique carried out for example on a microarray or by quantitative polymerase chain reaction.

One embodiment of the present disclosure comprises methods for preparing a fraction for haplotype determination comprising providing a nucleic acid sample comprising chromosomal components and asymmetrically distributing the chromosomal components into a plurality of fractions, thereby preparing a fraction for haplotype determination. In some embodiments, asymmetric distribution of the chromosomal components comprises delivering unequal amounts of the chromosomal components to different fractions of a plurality of fractions. In some embodiments, the ratio of asymmetrically distributed chromosomal components is not the same as the ratio of chromosomal components in the original population of cells. In some embodiments, asymmetric distribution of chromosomal components comprises differentially degrading chromosomal components in different fractions of a plurality of fractions. In some embodiments, asymmetric distribution of chromosomal components comprises differentially amplifying chromosomal components in different fractions of a plurality of fractions. In some embodiments, the nucleic acid sample is from a mammal, preferably a human. In other embodiments, the nucleic acid sample is from a non-human mammal, a plant or a virus. In some embodiments, the nucleic acid sample is from a plurality of cells, for example approximately 5 to 300 cells or approximately 10 to 100 cells. In some embodiments, the plurality of cells or metaphase synchronized while in other embodiments the plurality of cells is not metaphase synchronized. In some embodiments, the chromosomal components comprise two or more alleles at different loci wherein the alleles further comprise one or more sequences of interest.

One embodiment of the present disclosure comprises a method for determining the phasing of two or more sequences of interest comprising providing a fraction wherein the chromosomal components in the fraction are asymmetrically distributed, creating a library from the fraction, detecting a dateable signal for two or more sequences of interest in the library and determining the phasing of the two or more sequences of interest based on said differences in the detectable signal. In some embodiments, the detectable signal is a fluorescent signal. In some embodiments, the two or more sequences of interest are on the same chromosome and are further located at two or more different loci on the same chromosome. In some embodiments, the two or more different loci located on the same chromosome are separated by at least 10,000, at least 100,000, at least 100,000,000, or at least 200,000,000 nucleotides. In some embodiments, the fraction is from an individual organism. In some embodiments, the fraction is from a mammal, for example a human. In other embodiments, a fraction is from a non-human mammal, a plant or virus. In some embodiments, prior to providing a fraction for phase determination the degree of asymmetry between the two or more sequences of interest in the fraction is determined. In some embodiments, determining the degree of asymmetry comprises quantitative polymerase chain reaction analysis of the fraction. In some embodiments, determining the degree of asymmetry comprises microarray analysis of the fraction. In some embodiments, determining the degree of asymmetry comprises determining the signal-to-noise ratio between the two or more sequences of interest in the fraction. In some embodiments, the signal-to-noise ratio between the two or more sequences of interest in the fraction is greater than the signal-to-noise ratio in other fractions. In some embodiments, the signal-to-noise ratio is determined by fluorescence detection.

One embodiment of the present disclosure comprises methods for determining the phase of alleles at two or more different loci comprising providing an asymmetric distribution of nucleic acid molecules comprising alleles at two or more different loci, wherein the asymmetric distribution comprises a plurality of fractions, wherein the individual fractions comprise multiple copies of the alleles and wherein the individual fractions comprise different quantities of the alleles, distinguishing the alleles in the copies of the nucleic acid molecules that are present in one or more individual fractions, evaluating the different quantities of the alleles that are present in the one or more individual fractions, and determining the phase for the alleles at the two or more different loci from the distinguishing of the alleles and from the evaluating the different quantities of the alleles. In some embodiments, the evaluating comprises detecting differences in the number of fluorescent sequencing reads of the alleles at two or more different loci out of a total number of reads of the alleles at the two or more different loci. In some embodiments, the asymmetric distribution of nucleic acid molecules is from an individual organism. In some embodiments, evaluating the different quantities of the alleles comprises determining a ratio of alleles at the two or more different loci. In some embodiments evaluating the different quantities comprises counting alleles at the two or more different loci. In some embodiments, distinguishing the alleles comprises a nucleic acid sequencing technique, whereas in other embodiments distinguishing the alleles comprises a genotyping technique carried out on a microarray. In particular cases, a nucleic acid sequencing technique and an array-based genotyping technique can be used. In some embodiments, the two or more different loci are on the same chromosome and are separated by at least 10,000 nucleotides. In some embodiments, the two or more different loci located on the same chromosome are separated by at least 100,000, at least 100,000,000, or at least 200,000,000 nucleotides.

### DEFINITIONS

As used herein, the term “haplotype” refers to a haploid genotype, a combination or set of alleles or DNA sequences found at different locations or loci on a chromosome which are typically inherited as a unit and are linked, for example during a translocation event. A haplotype can provide a distinctive genetic pattern of an individual. A haplotype can be determined for one locus, several loci, or an entire chromosome depending on the number of recombination events that occur between a given set of loci. Alleles or DNA sequences are not limited to any specific type and include, for example, normal genetic sequences (i.e., non-variant) or variant genetic sequences. For example, single nucleotide polymorphisms (SNPs), short tandem repeats (STRs), etc. can be considered variant genetic sequences. The term “phased alleles” refers to the distribution of the particular alleles on a single chromosome. Accordingly, the “phase” of two alleles can refer to a characterization or determination of whether the alleles are located on a single chromosome or two separate chromosomes (e.g., a maternally or paternally inherited chromosomes). Unless otherwise stated, “haplotype” and “phased alleles” are considered synonymous.

As used herein, the term “isolated”, “purified” or “to purify” refers to the product or act of removing components (e.g., contaminants) from a sample. For example, nucleic acids are separated or isolated away from cellular debris or isolation reagents by removal of contaminating host cell or other proteins, salts, enzymes, buffers and the like used in isolating the nucleic acid from its present environment.

As used herein, the term “sample” is used consistent with its meaning in the art of biology and chemistry. In one sense, it is meant to include a nucleic acid from a specimen or culture obtained from any source such as biological and environmental samples. Biological samples may be obtained from animals including, but not limited to humans, non-human primates, and non-human animals including, but are not limited to, vertebrates such as rodents, ovines, bovines, ruminants, lagomorphs, porcines, caprines, equines, canines, felines, ayes, etc. Biological samples include, but are not limited to, fluids such as blood products, tissues, cells, and the like. Biological samples can further be of plant origin, monocotyledonous or dicotyledonous, deciduous or evergreen, herbaceous or woody, including but not limited to agricultural plants, landscape plants, nursery plants, and the like. Environmental samples may be bacterial, viral, fungal, and the like, in origin. Preferred samples are eukaryotic in origin. Basically, any organismal nucleic acid sample source of interest to an investigator in determining phased alleles is amenable to the present invention. A sample can also include a synthetic nucleic acid. Derivatives or products of nucleic acids such as amplified copies or chemically modified species are also included.

As used herein, the term “nucleic acid” can be, for example, a polymer of nucleotides, or a polynucleotide. The term can be used to designate a single molecule, or a collection of molecules. Nucleic acids may be single stranded or double stranded, and may include coding regions and regions of various control elements, non-coding regions, whole chromosomes, partial chromosomes, fragments and variants thereof.

As used herein, the terms “asymmetric”, “unbalanced”, “unequal” or “biased”, when used in reference to a distribution of like items, are considered synonymous unless otherwise stated. The terms refer to a collection of like items, for example chromosomes or chromosomal components, which are distributed across a plurality of fractions, aliquots, subsets, etc. such that different quantities of the like items occur at two or more individual fractions. Two or more of the individual fractions in the plurality of fractions can have like items. However, not all of the fractions in the plurality of fractions need to have an item; rather one or more fraction, aliquot, subset, etc. may have no items. An individual fraction can be homogeneous with respect to the items that are present or, alternatively, a heterogeneous collection of items can be present at an individual fraction such that multiple like items are present along with one or more dissimilar items. The like items can be substantially similar or identical. For example, the like items can be chromosomes that have a common sequence, fragments of chromosomes that have a common sequence, copies of at least a portion of a chromosome that have a common sequence or other nucleic acid molecules that have a common sequence. An asymmetric or unbalanced sample of like items can be made by discretizing the sample into fractions, aliquots, subsets, etc. whose ratio of components is not the same as the ratio in the original population. An asymmetric distribution of like items is, for example, a distribution of two parental chromosomal contributions (e.g., one maternally derived chromosome and one paternally derived chromosome) resulting in an unequal distribution, for example a 0.5:1, 1:1.5, 1:2, 1:3, 2:3, etc. ratio of the two parental chromosomal contributions in a fraction. A fraction, aliquot, subset, etc. can be, for example, a tube, well (e.g. in a microtiter plate), feature in a microarray, spot on a surface or substrate, bead, or particle, etc.

It will be understood that an asymmetry, imbalance or bias in a sample can be a relative characteristic or can be determined in a relative way. For example, a sample can have an asymmetry, imbalance or bias in chromosomes or chromosomal components that is characterized by a quantity of chromosomes or chromosomal components that is different from the quantity of chromosomes or chromosomal components present in an individual, tissue or cell from which the sample was derived. A such, it will be understood that an individual, tissue or cell from which a sample is derived can have a naturally occurring asymmetry, imbalance or bias in the quantity of at least one chromosome or chromosomal component, whereas the sample can be skewed to have a non-naturally occurring asymmetry, imbalance or bias in the quantity of the at least one chromosome or chromosomal component.

### FIGURES

FIG. 1 shows embodiments for generating pools of genetic materials comprising an unbalanced distribution of maternal and paternal chromosomal components.

FIG. 2 shows an example of a mixed population of chromosomes from both parents and the challenges in determining a haplotype from the mixed population.

FIG. 3 shows exemplary chromosomal populations and their use in determining a haplotype.

FIG. 4 demonstrates exemplary genotyping information available for practicing methods described herein comprising an unbalanced distribution of genetic material.

FIG. 5 demonstrates an exemplary loading percentage (expected number of target molecules loaded/assay well or location×100) versus the probability of generating useful information from a given assay (i.e., the probability of a measurable difference).

FIG. 6 demonstrates embodiments for methods of biased amplification for generating an unbalanced distribution of genetic material with two representative alleles, allele A and allele B.

FIG. 7 demonstrates examples of methods for biased degradation of templates for generating an unbalanced distribution of genetic material.

FIG. 8 demonstrates embodiments for methods of biased degradation for generating an unbalanced distribution of genetic material with two representative alleles, allele A and allele B.

FIG. 9 shows an exemplary scatterplot of fluorescence raw intensities of a normal diploid individual and the ability of the methods described herein to resolve heterozygous SNPs into their haploid components.

FIG. 10 shows a series of exemplary scatterplots of fluorescence raw intensities of two loci arbitrarily designated A (on the Y axis) and B (on the X axis) from 6 of the 12 diluted samples derived from the diploid sample of FIG. 9.

FIG. 11 shows aligned segments from a pool of unbalanced genetic material derived from cells HG01377 (top) and NA18507 (bottom) in the top panel and the merged haplotype blocks in the bottom panel (HG01377 and NA28507, respectively).

FIG. 12 shows aligned segments from a pool of unbalanced genetic material from a whole human genome of a normal individual derived from cells NA18506 (top panel) and the merged haplotype blocks in the bottom panel.

### DETAILED DESCRIPTION OF EMBODIMENTS

Embodiments of the present disclosure provide methods and systems for determining the haplotype of a biological sample. Particular embodiments provide methods for long range haplotyping of a genome. The importance of haplotyping a genome has far reaching implications, for example, in contributing to and driving a system of personalized healthcare, and contributing to successful organ and tissue transplants.

Conventional genotyping methods (e.g., microarray, sequencing, PCR, etc.) face difficulties in determining the haplotype of a single chromosome, particularly when the sequences of interest are located far apart on a chromosome. For example, microarray and PCR analyses as currently practiced do not typically provide haplotyping information, just the presence or absence of sequences. First generation sequencing techniques as currently practiced, such as capillary based methods of sequence analysis, may be able to detect sequences of interest that are proximal, for example within 1,000 bp or less depending on the system. Next generation sequencing as currently practiced falls somewhere in between as the scalability of next-generation sequencing (NGS) methods with regard to determining long-range haplotypes has been limited by relatively short sequencing reads (e.g., a few hundred base pairs depending on the system). Embodiments described herein fill the gap left by these aforementioned technologies by providing for the phasing of adjacent or proximal and distal or long-range alleles in a genome. Indeed, embodiments described herein are uniquely suited to identifying long-range haplotypes. The methods are particularly well suited for identifying haplotypes having a range that is longer than the length of nucleic acid fragments that are detected in a particular technique that is used. For example, NGS-based embodiments of the methods set forth herein can be used to identify haplotypes having a range that is longer than the read length of the NGS technique employed. The information gained from the phased alleles as provided by practicing the methods described herein finds utility in, for example, disease detection and personalized healthcare (PHC). For example, an individual's haplotype can be correlated to drug metabolism, drug discovery, disease states, cancers, disorders, transplant rejection risk, and the like. Indeed, with regards to personalized healthcare once a subject's phased haplotype is known then subject specific disease correlation and therapeutic options can be designed specifically to meet the needs of that subject.

Embodiments described herein provide superior alternatives compared to other methods for haplotyping. The present disclosure provides methods that are, for example, easy to use, are amenable to high-throughput applications, and have the ability to phase long-range alleles regardless of whether the sample is haploid or diploid, and regardless of whether a sample is homozygous or heterozygous for the alleles of interest.

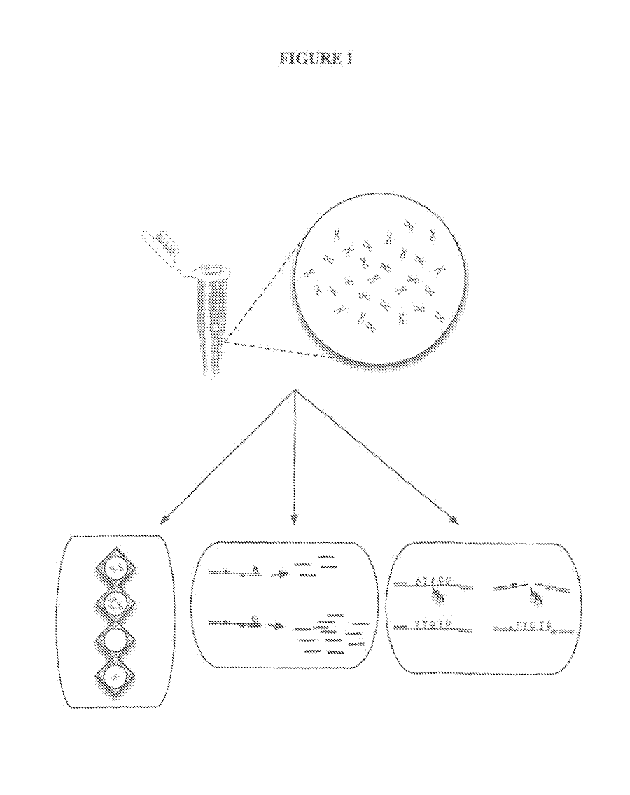

Embodiments for generating pools of genetic material for haplotype determination are exemplified in FIG. 1. One embodiment of a method for generating pools of genetic material with unbalanced distribution of maternal and paternal chromosomal components for a large portion of a genome or chromosome(s) comprises taking advantage of Poisson randomness to produce unequal distribution of genetic material (left arrow). For example, a normal DNA sample has a 1:1 ratio of maternal to paternal chromosomes. That sample can be fractioned by practicing methods disclosed herein to yield other than a 1:1 ratio, for example at least a 1:0.5, at least a 1:2, at least a 1:3, at a least 1:4, at a least 2:1, at least a 2:3, etc. of maternal to paternal chromosomes (or vice versa), hence an unbalanced distribution of chromosomes.

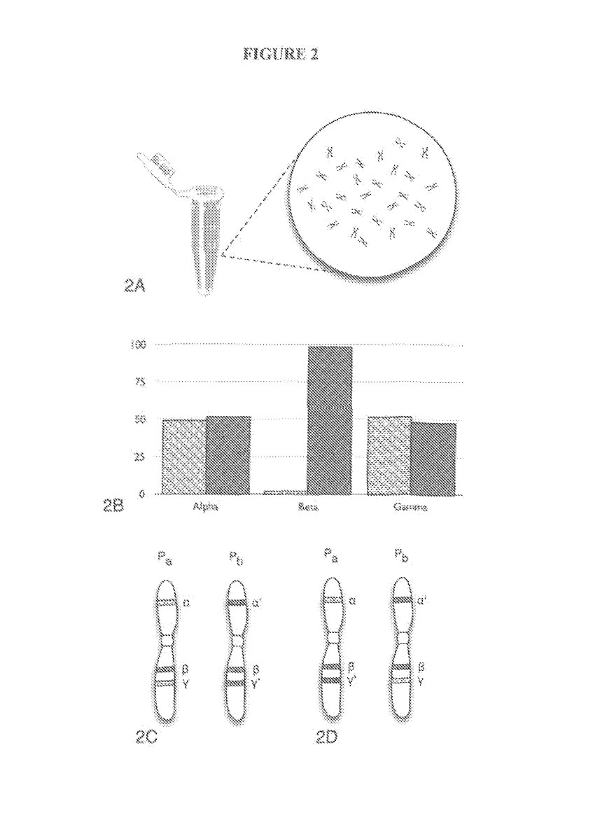

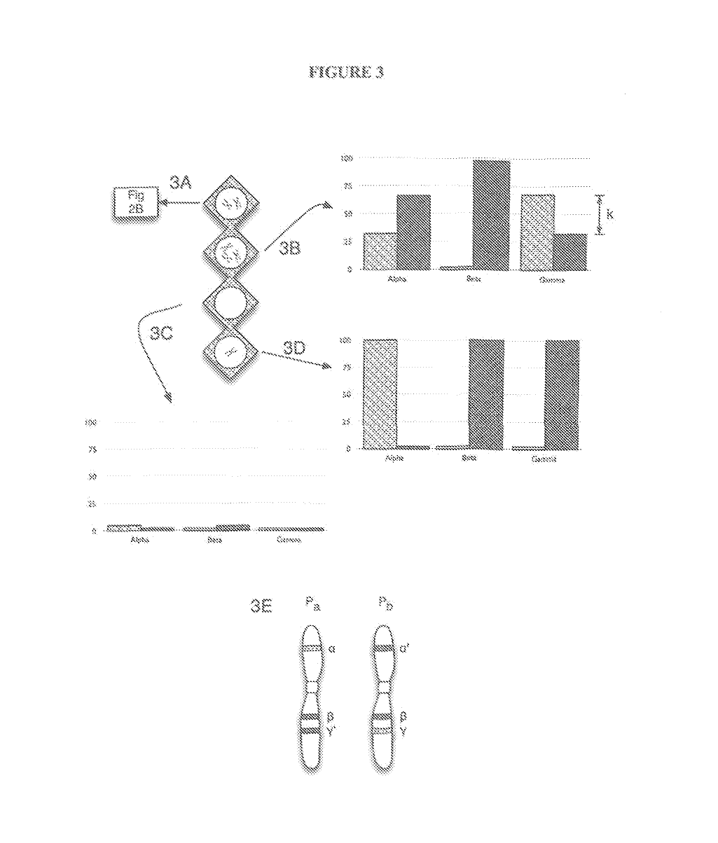

Embodiments of the present disclosure comprising taking advantage of Poisson randomness to produce unequal distribution of genetic material are exemplified in FIGS. 2 and 3. A genotyping sample may consist of a mixed population of chromosomes from both parents (FIG. 2A). While it is possible to determine the genotype for the patient (FIG. 2B), this type of analysis will not show how heterozygous alleles are grouped together on the chromosome. In this example, it is unknown whether Parent A is providing both exemplary (−) alleles at genes alpha and gamma, and Parent B the exemplary (+) ones (FIG. 2C), or if they are mixed (FIG. 2D). One method to determine the haplotype comprises isolating each chromosome into its own compartment (FIG. 3D) and treat it as a separate sample. In this way, each sample is homozygous at all alleles since there is only one copy of each gene in the compartment. However, the disadvantages of this method are that there will be many empty assay wells (FIG. 3C) (however, empty wells can be advantageous for use as a negative assay control) and that the signal from a well with a single chromosome may be very low. The methods set forth herein provide for chromosomal samples in fractions such as assay wells or compartments at higher concentrations and asymmetrically distributed across those fractions. As long as there is an unequal number of chromosomes (or nucleic acid molecules having sequences derived from the chromosomes) from each parent (FIG. 3B), for example as contrasted to FIG. 3A which shows an equal number of parental chromosomes, alleles from the chromosome with the greater number can display a higher detection signal (e.g., fluorescence, luminescence, etc.), and thereby be associated with each other, allowing for determination of haplotype of the different chromosomes (FIG. 3E).

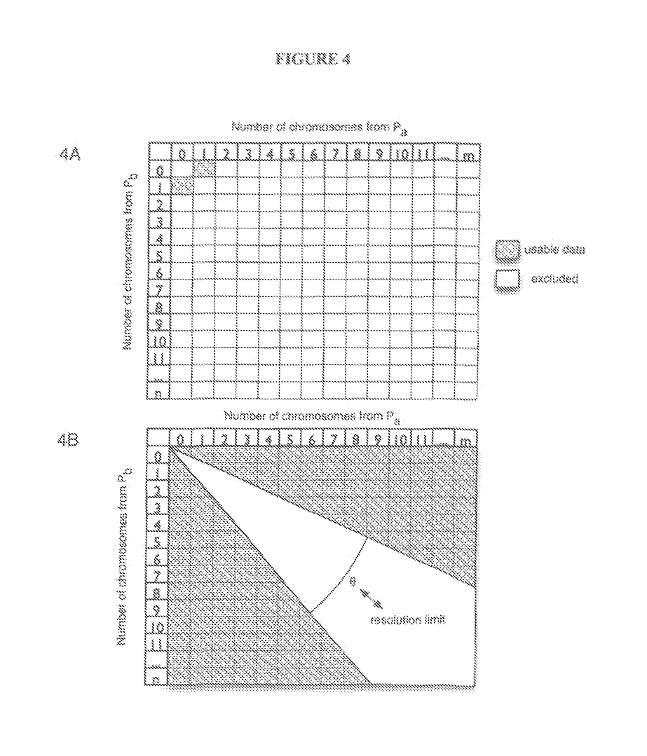

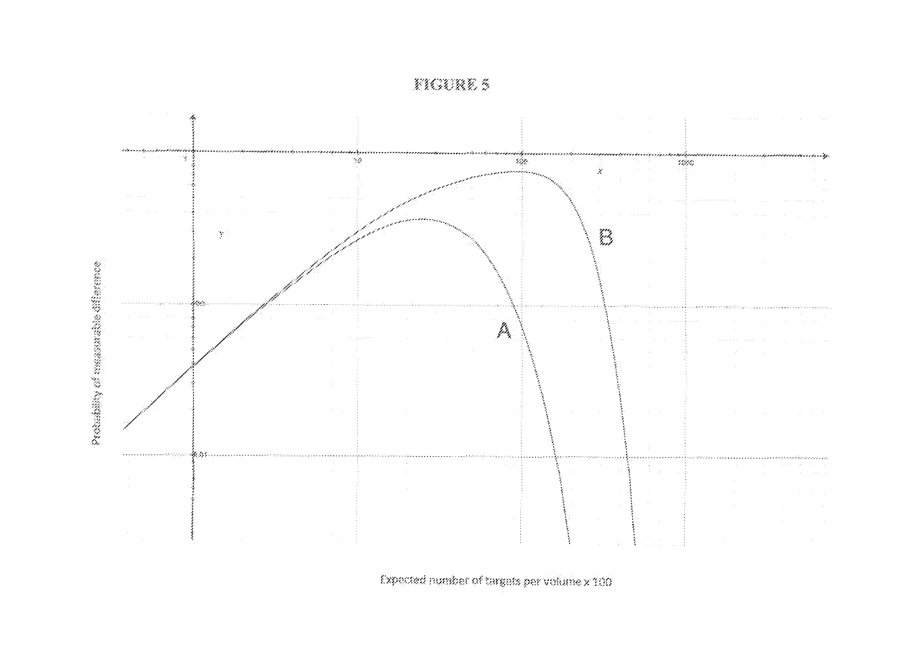

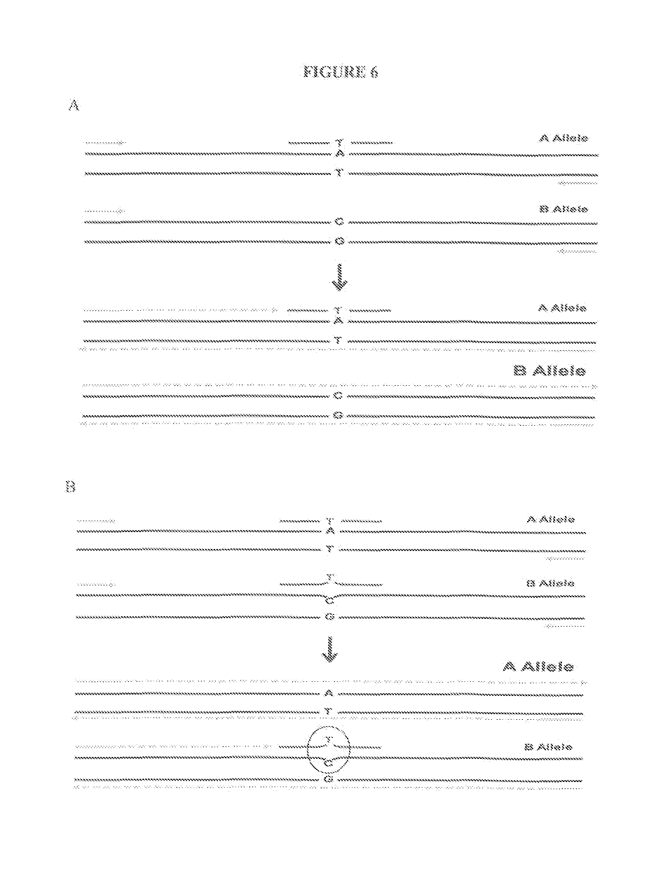

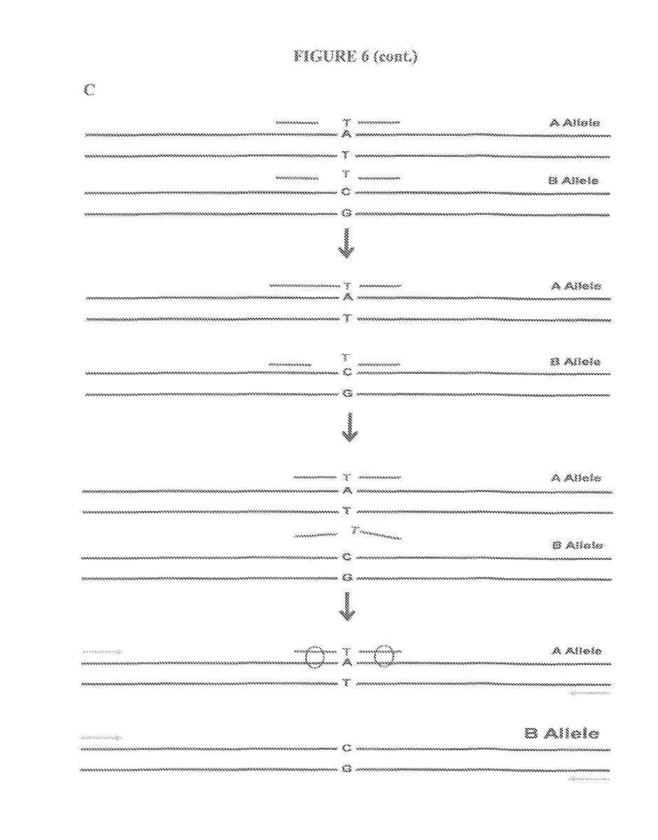

It is contemplated that the estimated improvement of practicing particular methods of the present disclosure can result in a 2-3× increase in loading density and 5-6× increase in total useable data from a given assay (FIGS. 4 and 5) compared to existing technologies. For example, FIG. 4A demonstrates the extent of genotyping information available from a standard dilution assay, wherein chromosomes are diluted down to single-molecule levels in the assay. Only those assay wells where one chromosome is present will provide useful data, for example Pₐ=1, P_{b}=0 or vice versa. Conversely, a large increase in the amount of useful information results from practicing embodiments of methods described herein since, for example, any number of chromosomes per volume can be used as long as the detection difference between the two different alleles is greater than the measurement threshold theta (θ)(FIG. 4B).

Since practicing embodiments of the disclosed method can result in both a greater density of loading and a higher probability of generating data per volume or fraction for a given number of fractions, the coverage of the haplome (i.e., haploid genome) will be higher compared to practicing other methods, such as the 0 to 1 dilution method (FIG. 5). For example, the maximum for a 0-or-1 dilution case (for example as exemplified in FIG. 5A) can be found at 24% loading with only 36% of the assay wells producing usable data. Alternatively, FIG. 5B demonstrates that an asymmetric loading method as disclosed herein can provide up to 100% loading with 76% of assay wells producing usable data. The resolution, or sensitivity, of a detection system is contemplated to affect the number of assay fractions that are needed to provide usable data. Target molecules (i.e., chromosomal components) comprise whole chromosomes, fragments of chromosomes, cloned chromosomal inserts such as those found in BACs, YACs, MACs, fosmids, cosmids, etc. Further, the disclosed methods can potentially provide equivalent coverage of the haplome with fewer fractions as compared to the 0 to 1 dilution method.

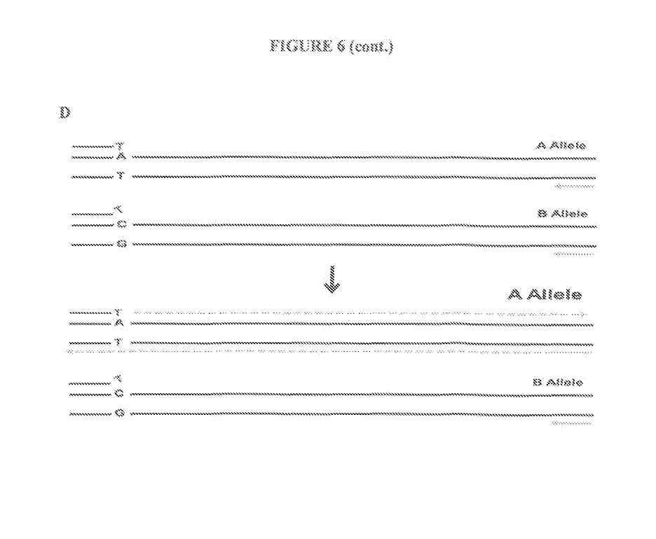

In one embodiment, a biased or unbalanced amplification method comprises primers and/or amplification conditions for amplifying alleles with different efficiencies such that one set of phased alleles is distinguishable in the amplified population is contemplated for generating an unbalanced distribution of genetic material (FIG. 1, middle arrow). Biased or unbalanced amplification such as biased or unbalanced polymerase chain reaction (PCR) can be used to generate an unbalanced distribution of two alleles by, for example, blocking (partially) the amplification of one of the alleles. For example, one embodiment comprises the use of blocking probes such as described in Rex et al. (2009, J. Virol. Meth. 158:24-29) and Senescau et al. (2005, J. Clin. Micro. 43:3304-3308) (both of which are incorporated herein by reference in their entireties). For example, a blocking probe can be the complement to one of the alleles (FIG. 6A, top reaction; blocking probe shown spanning the A nucleotide), has a Tm that is compatible with the extension temperature of the PCR, and has a 3′ blocking group preventing its elongation by DNA polymerase. Once the DNA polymerase (e.g., non-strand displacing) encounters the probe, strand elongation halts resulting in reduced representation of one allele in the final PCR product mixture. Conversely, strand elongation of the other allele will not be impeded by the presence of a blocking probe thereby resulting in a normal representation of that allele in the final PCR product mixture thereby resulting in a biased representation of one allele in the PCR product mixture (FIG. 6A, more allele B than allele A).

In another embodiment, a biased or unbalanced amplification method comprises a thermostable MutS protein and an allele-specific probe, for example an allele specific blocking probe, in an amplification reaction to create an unbalanced pool of genetic material (FIG. 6B). MutS is a DNA mismatch-binding protein that binds strongly to heteroduplex DNA in the presence of Mg²⁺ (Lishanski et al., 1994, Proc. Natl. Acad. Sci. 91:2674-2678; Stanislawska-Sachadyn and Sachadyn, 2005, Acta Biochim. Pol. 52:575-583; both of which are incorporated herein by reference in their entireties). For example, an allele specific blocking probe that is the complement of one allele can anneal to template DNA molecules forming both homoduplex DNA and heteroduplex DNA with the two allelic templates. MutS can preferentially bind to the blocking probe that has paired with the non-complement allele (FIG. 6B top reaction; heteroduplex formation shown on B allele and MutS binding shown as circle in bottom reaction). By using a strand-displacing DNA polymerase (e.g., phi29 DNA polymerase, BST DNA polymerase Large fragment, Vent® (exo−) DNA polymerase, Deep Vent® (exo−) DNA polymerase, 9° Nₘ DNA polymerase, etc.) the probe that is not bound by MutS can be removed (e.g., by negative antibody selection using anti-MutS) to allow for strand elongation of the perfect match template molecule whereas the MutS-complexed probe remains in place thereby halting strand elongation of the mismatched template molecule thereby producing an unbalanced representation of alleles in the final product mixture (FIG. 6B, more allele A than allele B).

In another embodiment, a biased or unbalanced amplification method is exemplified by FIG. 6C. In FIG. 6C (top set of alleles) short probes can be hybridized to either side of a locus. For those probes matched to specific alleles, extension and ligation of the probe can occur. However, when the probe and the allele are non-homologous, there is no or minimal extension and ligation (second from top set of alleles) of the probe. Following extension and ligation, the temperature can be raised such that those probes that have been extended and ligated will remain hybridized to the template whereas the short probes that have not been extended will be released from the template (third set of alleles). The hybridized and extended probes can be crosslinked to the template, thereby blocking PCR amplification resulting in more of one allele than the other (in this case, more allele B than allele A).

In another embodiment, a biased or unbalanced amplification method is exemplified By FIG. 6D. FIG. 6D shows the use of allele specific PCR wherein one of the primers anneals near a polymorphic site (i.e., location of a SNP or other polymorphism) at it 3′ end. The mismatched primer will not initiate replication whereas the matched primer can replicate as such resulting in more of one allele than the other (FIG. 6D, more allele A than allele B) (Newton, 1989, Nucl. Acid. Res. 17:2503-2516; incorporated herein by reference in its entirety).

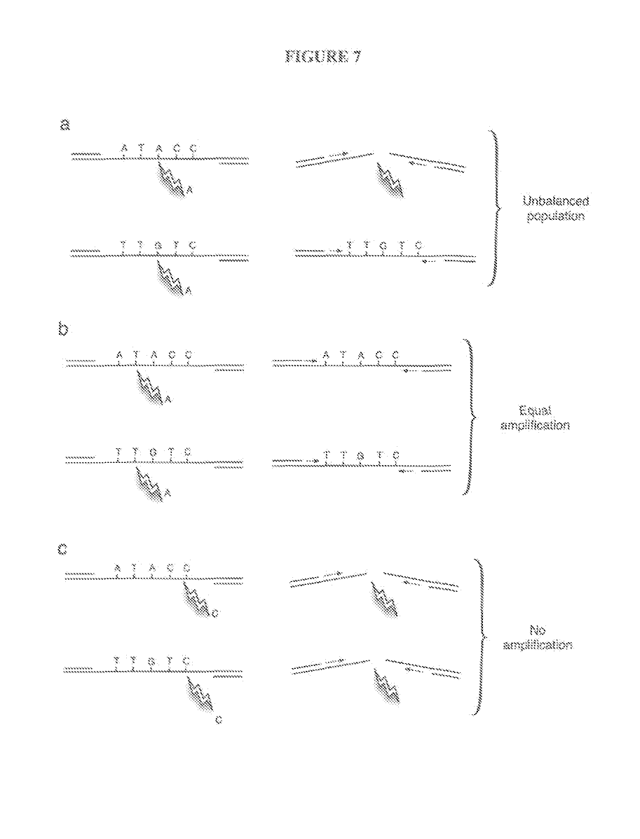

In one embodiment, generating an unbalanced distribution of genetic material comprises biased degradation of an allele (FIG. 1, right arrow). For example, templates can be digested at an allele-specific location on two loci (e.g., exemplary loci comprising ATACC and TTGTC) between the primers such that only one allele (e.g., the undigested allele) amplifies and all the alleles on the amplified strand therefore share the same phase (FIG. 7). A sample can be split into several separate fractions (A, B and C). Some loci will be heterozygous (7A) at the allelic target (A and G) wherein after degradation the resulting population will be over representative of a single haploid component (in this example locus TTGTC and allele G), thereby allowing for phasing of all alleles in the region after, for example, indexing and sequencing the separate reactions. Some loci will be homozygous (for example, 7B and C) at the allelic target (allele T), producing either an equally amplified population between the two haploid chromosomal contributions (7B) or little or no amplification (7C, allele C).

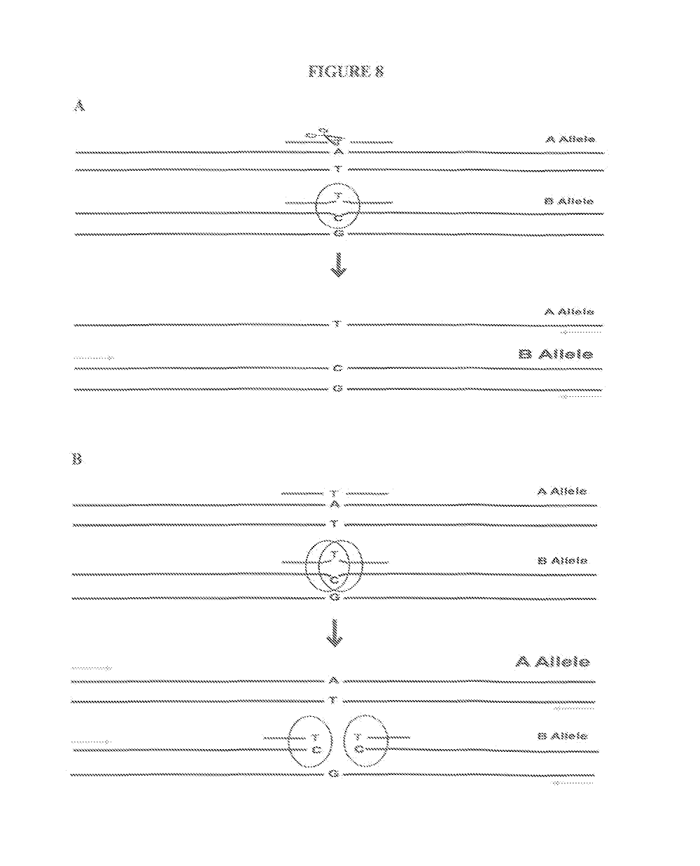

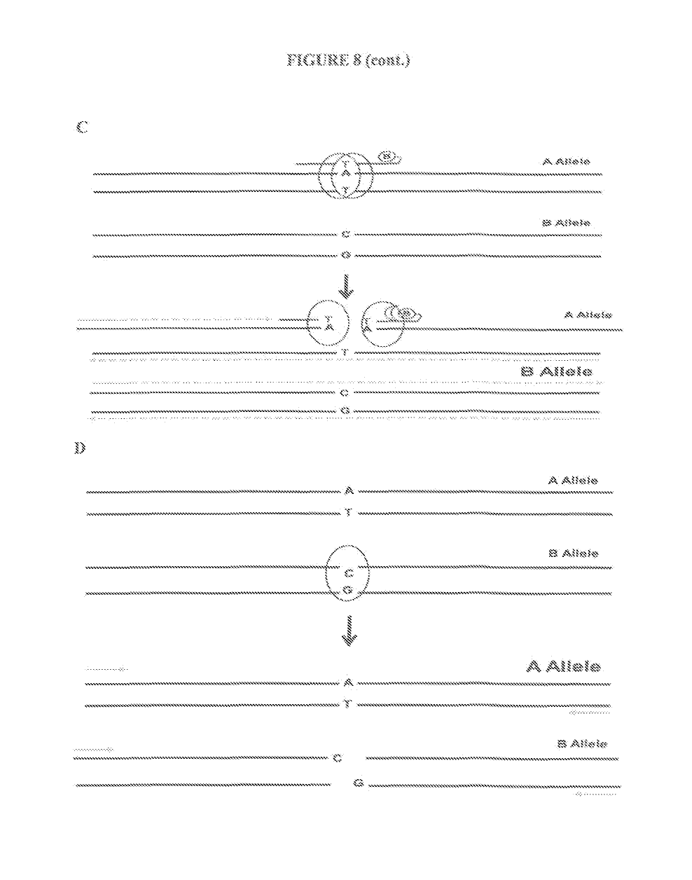

FIG. 8 demonstrates several exemplary embodiments for methods of biased degradation. As an exemplary modification of FIG. 6B, FIG. 8A demonstrates that a perfect match duplex molecule could be selectively destroyed with, for example, Duplex-Specific Nuclease, DSN, while the MutS-bound mismatched duplex is protected from cleavage. FIG. 8A demonstrates use of a thermostable MutS protein (circle), an allele specific probe and a duplex specific nuclease (scissors), wherein a duplex specific nuclease can cleave the homoduplex DNA for biased amplification of allele B over allele A.

In another embodiment, a biased degradation method comprises a phage Mu transposon that has a strong target site preference for single-nucleotide mismatches (Yanagihara and Mizuuchi, 2002, Proc. Natl. Acad. Sci. 99:11317-11321; incorporated herein by reference in its entirety) and an allele-specific probe. Mu can preferentially insert itself in heteroduplex DNA with a mismatch such that its use in, for example, a library preparation protocol (FIG. 8B, Mu transposon shown as circles) could serve to fragment template molecules of the mismatch allele whereas template molecules of the perfect match allele remain intact and serve as template in the PCR amplification thereby creating a biased or unbalanced genetic pool for haplotype determination (FIG. 8B, more allele A than allele B).

In another embodiment, a biased or unbalanced amplification method is exemplified by FIG. 8C, which is a modification of FIG. 8B. In FIG. 8C, a biotinylated allele specific probe (with B) is shown to hybridize to the template DNA. A streptavidin transposon fusion protein (for example, a Mu transposon as exemplified in the Nextera DNA sample prep kit from Epicentre Biotechnologies designated by circles) can be recruited to the double stranded hybridization site through the streptavidin-biotin interaction thereby resulting in fragmentation of the perfect match allele and more of one allele than the other (FIG. 8C, more of allele B than allele A).

In another embodiment, a biased degradation method can comprise a restriction endonuclease as demonstrated in FIG. 8D. For example, one or more restriction endonucleases may be chosen such that there will be approximately one restriction site per amplicon pair (e.g., by targeting known heterozygous loci or by statistics based on amplicon length). The amplicon comprising the targeted site can be degraded (i.e., restricted by restriction endonuclease designated at the circle) such that amplification is not possible. The undigested allele can be preferentially amplified yielding an unequal representation of alleles for haplotype determination (FIG. 8D, more allele A than allele B).

The present disclosure provides methods for determining the haplotype of a genome. In one embodiment, methods of the present disclosure create an unbalanced distribution of genetic material (i.e., chromosomal components) from a diploid or haploid genomic sample from a subject. Genotyping the unbalanced genetic material with standard methods (e.g., microarray, sequencing, PCR, gel based, etc.) allows the determination of haplotype over large genomic regions for long-range haplotyping. For example, when utilizing methods as described herein for asymmetric or unbalanced distribution of genetic material for haplotyping, if one set of target sequences of interest within a certain genomic region(s) are 3× higher in amplification signal intensity (e.g., via microarray) or 3× more reads (sequencing) than another set of alleles, then it is inferred that the two respective sets correspond to two distinct haplotypes. The relative amount of each target sequence of interest in the unbalanced genetic material pool, once determined, is compared to the amount determined from a normal diploid genome or pooled normal genomes thereby determining anomalies within the test sample.

The present disclosure provides methods comprising an unequal, unbalanced, biased or asymmetric distribution of a sample for haplotype determination. The unequal distribution can be the result of, for example, dilution, asymmetric PCR, targeted degradation, etc. In particular, embodiments described herein provide for genetic material from a subject to be distributed unevenly between fractions, such as assay locations on a substrate (e.g., wells in a plate, areas on a slide, a plurality of capillary tubes, wells in/on a flexible tape, etc.). In certain embodiments, the uneven distribution of genetic material of a sample represents an unequal distribution of chromosomes located at one or more assay locations on a substrate. It is contemplated that some assay locations will contain no genetic material and these locations find utility as negative controls within an assay as exemplified in FIG. 3C. Substrates include, but are not limited to, microarray substrates such as silica or high density plastic slides, chips, and the like, plates such as 96, 384, 1536 well assay plates, capillary tubes for example as used for flow through PCR, flexible high throughput assay strips (e.g., Array Tape™ by Douglas Scientific), beads, nanoparticles, etc. Methods described herein are not limited by the substrate upon which, or in which, an assay is performed.

Particular embodiments of methods described herein can be used, for example, to determine haplotypes of sequences of interest that are both proximal and distal to one another on a chromosome. It is contemplated that the sequences of interest are not separated by any particular distance, for example the sequences of interest may be adjacent, or proximal, to each other on a chromosome. Conversely, it is contemplated that the sequences of interest are distally separated, or long-range, from each other on a chromosome. Indeed, practicing embodiments described herein can be particularly beneficial in determining long-range haplotypes. Distances between the sequences of interest are not intended to limit the methods, for example the sequences of interest can be separated by at least 100, 200, 300, 400, 500, 750, or at least 1000 base pairs. However, embodiments find particular utility for determining haplotypes for sequences of interest when they are spaced far apart on the chromosome and are separated by, for example, at least 10,000, at least 100,000, at least 1,000,000, at least 10,000,000, at least 100,000,000, at least 150,000,000, at least 200,000,000, at least 247,000,000 or more base pairs. As such, embodiments described herein can provide methods particularly suited for long-range haplotyping of an individual genome regardless of whether the sample for determination is provided haploid or diploid.

In embodiments of the present disclosure, methods for determining haplotypes, particularly sequences of interest that are distally located on a chromosome are provided. In some embodiments, the sequences of interest are single nucleotide polymorphisms, or SNPs. In some embodiments, the SNPs are adjacent, or proximal to each other, while in other embodiments the SNPs are distal, or long-range, to each other. In some embodiments, the sequences of interest are insertions or deletions, or indels, of sequences within a genome. In some embodiments, the sequences of interest are genomic copy number variants, or CNVs. In other embodiments, the sequences of interest are alleles, or alternative forms of genes or sequences that are located at specific locations on a chromosome. In some embodiments, alleles are wild-type or normal, recognized sequence whereas in other embodiments alleles may harbor one or more mutations as compared to wild-type, such as SNPs, CNVs, indels, etc.

Such mutations may be identified to directly correlate to disease states, such as cancers, genetic diseases, and the like. Mutated alleles are of particular interest to investigators and practicing embodiments of the present disclosure can provide valuable tools in enabling investigators to study allelic mutations and their haplotypes. Haplotypes are valuable in defining the genetic makeup of a diploid genome of an individual. Haplotyping information can lead to greater understanding and find broader utility in many areas of scientific study, including but not limited to, drug metabolism, drug discovery, personalized healthcare initiatives, HLA typing for transplant success population genetics, complex disease linkage, genetic anthropology, medical genetics of diseases and cancers, structural variations in cancers and other diseases, allele specific expression and modifications such as allele specific methylation patterns and de novo genome assembly. Embodiments comprising biased amplification and biased degradation are particularly advantageous when the alleles of interest for haplotyping are from a small genomic region. As such, clinical applications such as HLA genotyping (e.g., HLA-A, HLA-B, HLA-C, HLA-DRB1, HLA-DQB1, HLA-DQA1, etc.), wherein haplotype determination over a few kilobases or one or more genomic regions is desired, would benefit greatly from practicing the methods disclosed herein.

The ability to assign alleles to chromosomes (i.e., haplotyping) is powerful because it can provide information of clinical relevance, for example by providing information about recombination events in the genome. Such information can be important for locating mutations that cause disease and can help determine linkage disequilibrium, or the statistical association between the presence of two polymorphisms in a genome; a key property of disease genome wide disease association studies. For example, knowing the genotype at one polymorphism (i.e., SNP) can help predict the genotype of another polymorphism (i.e., SNP) if the association (i.e., linkage disequilibrium) between the two polymorphisms is high. The ability to more completely match human leukocyte antigens (HLA) by determining their haplotypes would greatly improve the clinical outcome of, for example transplant recipients (Crawford and Nickerson, 2004, Ann. Rev. Med. 56:303-320, incorporated herein by reference in its entirety). For example, by practicing methods disclosed herein transplant recipients and potential donors could be genotyped at a plurality of markers along the major histocompatibility complex and the haplotypes could be determined from the generated data. Examples of such alignments can be found in Examples as disclosed herein. Such alignments could provide for highly accurate HLA matching between the transplant recipient and donor resulting in a better transplant outcome than patients and donors who are not so matched.

Additionally, there are some diseases wherein a haplotype and not a genotype at a particular locus can predict the severity of a disease as such an accurate haplotype would have wide utility for determining not only the severity of a disease for a particular patient, but also provide a clinician with information in determining potential treatment options based on that diagnosis and/or prognosis as different treatment options may correlate with different disease states and/or levels of severity. For example, a specific sickle-cell anemia β-globin locus haplotype is correlated with less severe sickle-cell anemia and a haplotype of an IL10 promoter region has been associated with lower incidence of graft-versus-host disease and death in patients receiving cellular transplants. As such, methods that would provide for haplotyping of a genomic sample could have a great impact on, for example, studies of disease correlation, disease diagnostic and prognostic practices, and application of therapeutic regimens. However, haplotyping is also of great importance in agriculture and other horticulture arts, particularly in the breeding of livestock and crop plants wherein diseases or advantageous properties could be correlated with particular haplotypes in an animal or plant.

Embodiments provided herein describe methods for the determination of phased alleles in a sample. Typically, a sample comprises a nucleic acid sample. In some embodiments, the nucleic acid sample is derived from a bodily fluid, for example blood, sputum, urine, spinal fluid, etc from a subject. In other embodiments, the biological sample is derived from a solid, for example a tissue, biopsy, cell scraping, cytology or cell sample, etc. from a subject. In one embodiment, the biological sample is a purified, single chromosome or fragments thereof, or a DNA insert for example in a cosmid, fosmid, plasmid, yeast artificial chromosome (YAC), bacterial artificial chromosome (BAC), mammalian artificial chromosome (MAC), plant cloning systems (e.g., Agrobacterium tumefacians T-DNA cloning systems, binary vector cloning systems, etc.), or fragments thereof, and the like. In preferred embodiments, the biological sample is a diploid DNA sample as found in one or more cells. However, embodiments of methods described herein are not limited to diploid samples as haploid samples (e.g., nucleic acid derived from an egg, sperm, hydatiform mole, and mechanically separated and/or isolated chromosomes, fragments thereof, cloned DNA fragments, etc.) are equally amenable to practicing the methods described herein.

In one embodiment, a sample is a cell sample or a tissue sample. A cell or tissue sample can be from any source, for example cells from a dissociated tissue, cells from blood or other body fluids, cells from a cytological specimen, cells from a non-human animal, cells from a plant, etc. In preferred embodiments, cells are mammalian in origin, preferably human in origin. However, methods described herein are not limited to the source of the cell sample. In some embodiments, the genomic material used in practicing methods described herein is derived from a plurality of cells. In some embodiments, the plurality of cells is at least between 2 and 1000 cells, at least between 5 and 500 cells, at least between 10 and 300 cells, at least between 10 and 100 cells. The practice of the methods set forth herein can employ, unless indicated specifically to the contrary, conventional methods of virology, immunology, microbiology, molecular biology and recombinant DNA techniques within the skill of the art. Such techniques are explained fully in the literature; see, e.g., 1995, Ausubel et al., Short Protocols in Molecular Biology, (3^{rd} ed.), Wiley & Sons; 2001, Sambrook and Russell, Molecular Cloning: A Laboratory Manual (3rd Edition); 1982, Maniatus et al., Molecular Cloning: A Laboratory Manual; DNA Cloning: A Practical Approach, vol. I & II (D. Glover, ed.); 1984, Oligonucleotide Synthesis (N. Gait, ed.); 1985, Nucleic Acid Hybridization (B. Hames & S. Higgins, eds.); 1986, Animal Cell Culture (R. Freshney, ed.); 1984, Perbal, A Practical Guide to Molecular Cloning. Genomic material can be harvested by methods known in the art and the methods described herein are not necessarily limited to any particular method for isolation of genomic material. A skilled artisan will understand that a myriad of commercial and homebrew alternatives exist for such isolation.

In one embodiment, a sample for haplotyping is provided by a subject. A subject can be any biological entity of interest to an investigator who wishes to determine a haplotype from that entity. As such, a sample for testing is not necessarily limited to a particular subject and a subject can be for example animal or plant in origin. For example, a subject providing a sample could be an animal, either human or non-human, or a plant, for example economically relevant crop plants and the like. In preferred embodiments, a subject is a human. In other preferred embodiments, a subject is an economically relevant animal or derivative thereof. In other embodiments, a subject is an economically relevant plant or derivative thereof.

The asymmetrically distributed samples provided by practicing the methods of the present disclosure are readily applied to downstream applications. In some embodiments, it is contemplated that downstream processes are performed on the samples prior to sequencing or other instrument related haplotype determination. In some embodiments, an aliquot or fraction of an asymmetrically distributed sample is used to prepare a DNA library for clustering for next-generation sequencing. Such a library is produced, for example, by performing the methods as described in the Nextera™ DNA Sample Prep Kit (Epicentre® Biotechnologies, Madison Wis.), GL FLX Titanium Library Preparation Kit (454 Life Sciences, Branford Conn.), SOLiD™ Library Preparation Kits (Applied Biosystems™ Life Technologies, Carlsbad Calif.), and the like. The sample as described herein is typically further amplified for sequencing or microarray assays by, for example, multiple stand displacement amplification (MDA) techniques. For sequencing after MDA, an amplified sample library is, for example, prepared by creating a DNA library as described in Mate Pair Library Prep kit, Genomic DNA Sample Prep kits or TruSeq™ Sample Preparation or Exome Enrichment kits (Illumina®, Inc., San Diego Calif.). Useful cluster amplification methods are described, for example, in U.S. Pat. No. 5,641,658; U.S. Patent Publ. No. 2002/0055100; U.S. Pat. No. 7,115,400; U.S. Patent Publ. No. 2004/0096853; U.S. Patent Publ. No. 2004/0002090; U.S. Patent Publ. No. 2007/0128624; and U.S. Patent Publ. No. 2008/0009420, each of which is incorporated herein by reference in its entirety. Another useful method for amplifying nucleic acids on a surface is rolling circle amplification (RCA), for example, as described in Lizardi et al., Nat. Genet. 19:225-232 (1998) and US 2007/0099208, each of which is incorporated herein by reference in its entirety. Emulsion PCR methods are also useful, exemplary methods which are described in Dressman et al., Proc. Natl. Acad. Sci. USA 100:8817-8822 (2003), WO 05/010145, or U.S. Patent Publ. Nos. 2005/0130173 or 2005/0064460, each of which is incorporated herein by reference in its entirety. Methods of the present disclosure are not necessarily limited by any particular library preparation or amplification method as an asymmetrically distributed sample as described herein is contemplated to be amenable to any of a variety of methods known in the art and/or commercially available for such purposes.

For example, DNA libraries comprising the unbalanced distribution of genetic material can be immobilized on a substrate, such as a flowcell, and bridge amplification performed on the immobilized polynucleotides prior to sequencing, for example sequence by synthesis methodologies. In bridge amplification, an immobilized polynucleotide (e.g., from a DNA library) is hybridized to an immobilized oligonucleotide primer. The 3′ end of the immobilized polynucleotide molecule provides the template for a polymerase-catalyzed, template-directed elongation reaction (e.g., primer extension) extending from the immobilized oligonucleotide primer. The resulting double-stranded product “bridges” the two primers and both strands are covalently attached to the support. In the next cycle, following denaturation that yields a pair of single strands (the immobilized template and the extended-primer product) immobilized to the solid support, both immobilized strands can serve as templates for new primer extension. Thus, the first and second portions can be amplified to produce a plurality of clusters. The terms “cluster” and “colony” are used interchangeably and refer to a plurality of copies of a nucleic acid sequence and/or complements thereof attached to a surface. Typically, the cluster comprises a plurality of copies of a nucleic acid sequence and/or complements thereof, attached via their 5′ termini to the surface. Exemplary bridge amplification and clustering methodology are described, for example, in PCT Patent Publ. Nos. WO00/18957 and WO98/44151, U.S. Pat. No. 5,641,658; U.S. Patent Publ. No. 2002/0055100; U.S. Pat. No. 7,115,400; U.S. Patent Publ. No. 2004/0096853; U.S. Patent Publ. No. 2005/0100900, U.S. Patent Publ. No. 2004/0002090; U.S. Patent Publ. No. 2007/0128624; and U.S. Patent Publ. No. 2008/0009420, each of which is incorporated herein by reference in its entirety. The compositions and methods as described herein are particularly useful in sequence by synthesis methodologies utilizing a flowcell comprising clusters.

Emulsion PCR methods for amplifying nucleic acids prior to sequencing can also be used in combination with methods and systems as described herein. Emulsion PCR comprises PCR amplification of an adaptor flanked shotgun DNA library in a water-in-oil emulsion. The PCR is multi-template PCR; only a single primer pair is used. One of the PCR primers is tethered to the surface (5′ attached) of microscale beads. A low template concentration results in most bead-containing emulsion microvesicles having no more than one template molecule present. In productive emulsion microvesicles (an emulsion microvesicle where both a bead and template molecule are present), PCR amplicons can be captured to the surface of the bead. After breaking the emulsion, beads bearing amplification products can be selectively enriched. Each clonally amplified bead will bear on its surface PCR products corresponding to amplification of a single molecule from the template library. Various embodiments of emulsion PCR methods are set forth, for example, in Dressman et al., Proc. Natl. Acad. Sci. USA 100:8817-8822 (2003), PCT Patent Publ. No. WO 05/010145, U.S. Patent Publ. Nos. 2005/0130173, 2005/0064460, and US2005/0042648, each of which is incorporated herein by reference in its entirety.

DNA nanoballs can also be used in combination with methods and systems as described herein. Methods for creating and utilizing DNA nanoballs for genomic sequencing can be found at, for example, U.S. Pat. No. and publications 7,910,354, 2009/0264299, 2009/0011943, 2009/0005252, 2009/0155781, 2009/0118488 and as described in, for example, Drmanac et al., 2010, Science 327(5961): 78-81; all of which are incorporated herein by reference in their entireties. Briefly, following genomic DNA fragmentation consecutive rounds of adaptor ligation, amplification and digestion results in head to tail concatamers of multiple copies of the circular genomic DNA template/adaptor sequences which are circularized into single stranded DNA (e.g. by ligation with a circle ligase) and rolling circle amplified (for example, as described in Lizardi et al., Nat. Genet. 19:225-232 (1998) and US 2007/0099208 A1, each of which is incorporated herein by reference in its entirety). The adaptor structure of the concatamers promotes coiling of the single stranded DNA thereby creating compact DNA nanoballs. The DNA nanoballs can be captured on substrates, preferably to create an ordered or patterned array such that distance between each nanoball is maintained thereby allowing sequencing of the separate DNA nanoballs.

In some embodiments, once the asymmetrically distributed sample is further processed it is applied to sequencing, microarray analysis, genotyping, or other downstream applications. For example, sequencing can be performed following manufacturer's protocols on a system such as those provided by Illumina, Inc. (HiSeq 1000, HiSeq 2000, Genome Analyzers, MiSeq, HiScan, systems), 454 Life Sciences (FLX Genome Sequencer, GS Junior), Applied Biosystems™ Life Technologies (ABI PRISM® Sequence detection systems, SOLiD™ System), Ion Torrent® Life Technologies (Personal Genome Machine sequencer) further as those described in, for example, in U.S. Pat. Nos. and patent applications 5,888,737, 6,175,002, 5,695,934, 6,140,489, 5,863,722, 2007/007991, 2009/0247414, 2010/0111768 and PCT application WO2007/123744, each of which is incorporated herein by reference in its entirety.

In some embodiments, methods described herein for determining a haplotype find particular utility when used in sequencing, for example sequencing by synthesis (SBS) technologies. Sequencing by synthesis generally comprises sequential addition of one or more labeled nucleotides to a growing polynucleotide chain in the 5′ to 3′ direction using a polymerase. The extended polynucleotide chain is complementary to the nucleic acid template, which can be affixed on a substrate (e.g., flowcell, chip, slide, etc.), and which contains the target sequence. The labeled nucleotides that are used in SBS can include any of a variety of fluorophores, mass labels, electronically detectable labels or other types of labels. The labeled nucleotides that are used in SBS can also include reversible terminator groups such that only one nucleotide is added per SBS cycle. After the incorporated nucleotide is detected a deblocking agent can be added to render the added nucleotide competent for extension in a subsequent cycle. SBS methods are particularly useful for parallel analysis of different-sequence fragments of a nucleic acid sample. For example, hundreds, thousand, millions or more different-sequence fragments can be sequenced simultaneously on a single substrate using known SBS techniques. Exemplary sequencing methods are described, for example, in Bentley et al., Nature 456:53-59 (2008), WO 04/018497; U.S. Pat. No. 7,057,026; WO 91/06678; WO 07/123,744; U.S. Pat. No. 7,329,492; U.S. Pat. No. 7,211,414; U.S. Pat. No. 7,315,019; U.S. Pat. No. 7,405,281, and US 2008/0108082, each of which is incorporated herein by reference in its entirety.

Disclosed methods for determining a haplotype also find utility when used in sequencing by ligation, sequencing by hybridization, and other sequencing technologies. An exemplary sequence by ligation methodology is di-base encoding (e.g., color space sequencing) utilized by Applied Biosystems' SOLiD™ sequencing system (Voelkerding et al., 2009, Clin Chem 55:641-658; incorporated herein by reference in its entirety).

The methods for haplotyping disclosed herein could be utilized in sequence by hybridization technologies. Sequence by hybridization comprises the use of an array of short sequences of nucleotide probes to which is added fragmented, labeled target DNA (for example, as described in Drmanac et al., 2002, Adv Biochem Eng Biotechnol 77:75-101; Lizardi et al., 2008, Nat Biotech 26:649-650, U.S. Pat. No. 7,071,324; incorporated herein by reference in their entireties). Further improvements to sequence by hybridization can be found at, for example, US patent application publications 2007/0178516, 2010/0063264 and 2006/0287833 (incorporated herein by reference in their entireties). Sequencing approaches which combine hybridization and ligation biochemistries have been developed and commercialized, such as the genomic sequencing technology practiced by Complete Genomics, Mountain View, Calif.). For example, combinatorial probe-anchor ligation, or cPAL™ (Drmanac et al., 2010, Science 327(5961): 78-81) utilizes ligation biochemistry while exploiting advantages of sequence by hybridization. Single molecule sequencing technologies, for example as described in Pushkarev et al. (2009, Nat. Biotechnol. 27:847-52; incorporated herein by reference in its entirety) and as practiced by HeliScope™ Single Molecule Sequencer (Helicos, Cambridge, Mass.) can also take advantage of the disclosed methods for determining a haplotype.

Methods as described herein are not limited by any particular sequencing sample preparation method and alternatives will be readily apparent to a skilled artisan and are considered within the scope of the present disclosure. However, particular utility is found when applying the methods herein to sequencing devices such as flow cells or arrays for practicing sequence by synthesis methodologies or other related sequencing technologies such as those practiced by one or more of polony sequencing technology (Dover Systems), sequencing by hybridization fluorescent platforms (Complete Genomics), sTOP technology (Industrial Technology Research Institute) and sequencing by synthesis (Illumina, Life Technologies).

In some embodiments, an asymmetrically distributed sample as described herein is processed by MDA and further processed for microarray and/or other genotype analysis assays. For example, in some embodiments the sample is processed by quantitative PCR (qPCR) to characterize individual fractions or aliquots for signal-to noise ratio (for example, by utilizing an Eco PCR system (Illumina®, Inc.)). Such characterization is useful in defining the fractions or aliquots that will potentially provide for the highest probability of interpretable data from downstream sequencing or microarray analysis. In some embodiments, further processing is performed for preparation prior to microarray analysis. For example, an asymmetrically distributed sample after amplification by MDA and/or characterization by qPCR is prepared for microarray analysis by a variety of methods, including but not limited to those previously described above for library sample preparation.

Exemplary microarrays that are useful include, without limitation, a Sentrix® Array or Sentrix® BeadChip Array available from Illumina®, Inc. (San Diego, Calif.) or others including beads in wells such as those described in, for example, U.S. Pat. Nos. 6,266,459, 6,355,431, 6,770,441, and 6,859,570 and PCT Publication No. WO 00/63437 (each of which is incorporated by reference in their entirety).

Other arrays having particles on a surface include those set forth in US 2005/0227252, US 2006/0023310, US 2006/006327, US 2006/0071075, US 2006/0119913, U.S. Pat. No. 6,489,606, U.S. Pat. No. 7,106,513, U.S. Pat. No. 7,126,755, U.S. Pat. No. 7,164,533, WO 05/033681 and WO 04/024328 (each of which is hereby incorporated by reference in its entirety). An array of beads useful in assaying an asymmetrically distributed sample as provided by practicing methods of the present disclosure can also be in a fluid format such as a fluid stream of a flow cytometer or similar device. Commercially available fluid formats for distinguishing beads include, for example, those used in XMAP™ technologies from Luminex or MPSS™ methods from Lynx Therapeutics.

Further examples of commercially available microarrays that can be used with samples provided by practicing methods of the present disclosure include, for example, an Affymetrix® GeneChip® microarray or other microarray synthesized in accordance with techniques sometimes referred to as VLSIPS™ (Very Large Scale Immobilized Polymer Synthesis) technologies as described, for example, in U.S. Pat. Nos. 5,324,633, 5,744,305, 5,451,683, 5,482,867, 5,491,074, 5,624,711, 5,795,716, 5,831,070, 5,856,101, 5,858,659, 5,874,219, 5,968,740, 5,974,164, 5,981,185, 5,981,956, 6,025,601, 6,033,860, 6,090,555, 6,136,269, 6,022,963, 6,083,697, 6,291,183, 6,309,831, 6,416,949, 6,428,752 and 6,482,591 (each of which is hereby incorporated by reference in its entirety).

A spotted microarray can also be used with a sample provided by practicing the methods of the present disclosure. An exemplary spotted microarray is a CodeLink™ Array available from Amersham Biosciences. Another microarray that is useful is one that is manufactured using inkjet printing methods such as SurePrint™ Technology available from Agilent Technologies. Other microarrays that can be used include, but are not limited to, those described in Butte, 2002, Nature Reviews Drug Discov. 1:951-60 or U.S. Pat. Nos. 5,429,807, 5,436,327, 5,561,071, 5,583,211, 5,658,734, 5,837,858, 5,919,523, 6,287,768, 6,287,776, 6,288,220, 6,297,006, 6,291,193, and 6,514,751 and WO 93/17126 and WO 95/35505 (each of which is hereby incorporated by reference in its entirety).

Output from a sequencing, microarray or other genotyping methodology or instrument can be of any sort. For example, some technologies utilize a light generating readable output, such as fluorescence or luminescence, whereas other technologies measure electrical or ion release. However, the present invention is not limited to the type of readable output as long as differences in output signal for a particular sequence of interest can be determined. Examples of analysis software that may be used to characterize output derived from practicing methods as described herein include, but are not limited to, Pipeline, CASAVA, Genome Studio Data Analysis, BeadStudio Genotyping and KaryoStudio data analysis software (Illumina®, Inc.), SignalMap and NimbleScan data analysis software (Roche NimbleGen), GS Analyzer analysis software (454 Life Sciences), SOLiD™, DNASTAR®SeqMan® NGen® and Partek® Genomics Suite™ data analysis software (Life Technologies), Feature Extraction and Agilent Genomics Workbench data analysis software (Agilent Technologies), Genotyping Console™, Chromosome Analysis Suite and GeneChip® Sequence Analysis data analysis software (Affymetrix®). A skilled artisan will know of additional numerous commercially and academically available software alternatives for data analysis for microarray, sequencing, and PCR generated output. Embodiments described herein are not limited to any data analysis method.

Exemplary methods of the present disclosure are not necessarily limited by any particular sequencing, microarray or genotyping system as the particular sample preparation required for a particular instrument is contemplated to be amenable for use with an asymmetrically distributed sample as described herein. However, it is contemplated that the resolution, or sensitivity, of any given detection system may influence the number of fractions that may be assayed to yield interpretable results. Resolution difference is exemplified in FIG. 3B (k) and FIG. 4B ( ).

The following example describes a method for determining SNP haplotype by sequencing utilizing an asymmetrically generated sample. In this particular example, a preparation method that utilizes low input DNA levels (e.g., 10-100 pg) such as the Nextera™ DNA Sample Prep Kit is particularly useful as samples processed by this kit are ready for sequencing and require no further processing, such as multiple stand displacement amplification. Otherwise, an additional amplification step, such as MDA, may be required. The prepared sample can be sequenced, for example on an Illumina, Inc. Genome Analyzer, HiSeq, MiSeq, TruSeq or other sequencing platform wherein a fluorescent readout corresponding to each fluorescently labeled nucleotide is produced for analysis. For purposes of example, the following sequencing result is obtained from an asymmetrically distributed sample preparation:

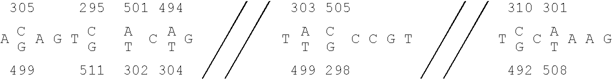

In this example, the nucleic acids for individual loci are separated from discontiguous and possibly distantly situated chromosomal regions by the double hash lines. The two nucleotides listed for one location represent heterozygous sequence variants, or single nucleotide polymorphisms (SNPs), in the sequences of interest. The numbers above and below the nucleotides represent the number of reads at that particular nucleotide location out of a total number of reads, for example in this case approximately 800 reads. Long range SNP phasing is determined by matching the SNP positions which have similar reads as follows:

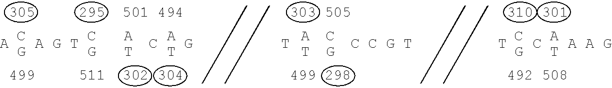

In this example, the circled numbers represent similar reads at different SNP positions and therefore determine which SNPs are located on the same chromosome or chromosomal fragment or segment and are therefore in phase, thereby determining the haplotype of the sample. As such, counting the number of reads for a plurality of SNPs and matching those read counts, can be used to determine the sample haplotype. The haplotype for the two chromosomal parental contributions (e.g., for example the top sequence is the maternal contribution and the bottom sequence is the paternal contribution) is determined to be:

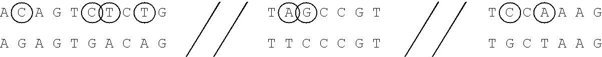

For purposes of explanation, haplotyping by microarray can be exemplified in a similar fashion, except in lieu of a nucleic acid sequence output a digitally derived color readout corresponding to an analog value of the computed hybridization intensity of each SNP, and the intervening sequences, can be provided.

In some embodiments, an asymmetrically distributed sample can be characterized prior to any additional processing, such as library preparation or amplification, or prior to library preparation utilizing the Nextera™ kit as previously exemplified. For example, a sample is fractioned or aliquoted, as found in Example 1, and each fraction is processed separately in sequencing or microarray analysis. As described in Example 1, if a sample is discretized into 10 fractions then 10 downstream processes can be run on one original sample. Separating a sample into multiple fractions or aliquots offers many advantages, including but not limited to, multiple analysis on one sample and lower cost and effort (e.g., reagents and other consumables, investigator time, etc.). To further reduce cost and effort, the multiple fractions can be characterized and/or quantified prior to analysis for those samples with the highest asymmetries, highest signal-to-noise ratio, and highest coverage of the desired target(s). For example, qPCR based genotyping of a fraction for a plurality of sequences would be sufficient to determine the asymmetry and signal-to noise ratio of a fraction. Further, microarray analysis or low depth sequencing methods can also be used to determine asymmetry and signal-to noise ratio of a fraction. Haplotype analysis of only those fractions with highest signal-to noise ratios, for example, are contemplated to provide the highest probability of yielding interpretable results, thereby saving time, effort and money.

A skilled artisan will appreciate that resolution of different detection systems used for haplotyping (e.g., sequencing, microarray analysis, qPCR, PCR, etc.) varies. Therefore, it is contemplated that the resolution limit of a given system should be taken into consideration when deciding the degree of asymmetry, signal-to-noise ratio, etc., that will provide the most useful data generated by any given system.

The following examples are provided in order to demonstrate and further illustrate certain embodiments and aspects of the present invention and are not to be construed as limiting the scope thereof.

### EXAMPLES

### Example 1

### Asymmetrical Distribution of a Sample

In evaluating methods for determining long range haplotyping of a genome, it was determined that distribution of a sample in such a way that the signal from the underlying chromosomal contributions (e.g., maternally derived chromosomal contribution and parentally derived chromosomal contribution) were distinguished one from another provided superior results in successful long-range haplotyping of a genome.

The following method is exemplary of how to generate an asymmetrical distribution of a sample that can be used for determining a long range haplotype. Chromosome 6 (Chr 6) is the exemplary chromosome, however it is understood that any chromosomally derived genetic material from any tissue, cell type, cell line, immortalized or primary, etc., can be used.

The sample contains a mixture of maternal and paternal contributions of Chr 6, referred to as M6 and P6, respectively. For purposes of this example, the sample is derived from cells synchronized to metaphase. Metaphase synchronization is not required to practice the described methods, however since this example demonstrates determining phased alleles for the two parental contributions one way to accomplish this is to begin with cells synchronized to metaphase.

A sample cell number is determined by fluorescent activated cell sorting or FACS, cytometric determination, or other known methods. Once sample cell number is determined, the sample is diluted to approximately 100 cells/μl in a final total cell volume of around 10 μl. Since on average a cell contains one copy each of M6 and P6, the ratio of the two contributions is 1:1. Following dilution, the cells are lysed and DNA harvested by established techniques known to a skilled artisan. For purposes of this example, the sample is divided into 10 fractions; discretizing the DNA sample into 10 fractions of 15 nl gives the optimal probability of a test sample containing 1.5 chromosomes (FIG. 5), providing a total number of potential test fractions of around 670. The final fraction volume will vary, for example depending on differences in cell concentration prior to DNA harvest, differences in target chromosomal components (e.g., in this case M6 and P6) and the sensitivity of the measurement technique used for downstream analysis (e.g., sequencing, microarray assays, PCR, etc.). The method for aliquoting or fractioning the sample can vary, for example for the purposes of this example a microfluidic device is used for fractioning the sample into a desired number of assay chambers for downstream applications, however any manual or automatic method of fractioning a sample is appropriate if the desired volume is within the range of that method.

If haplotype data from every chromosome is not required, for example if haplotyping data from a few chromosomes is desired, FIG. 5 can be used to determine the number of fractions required for downstream processing to yield the highest probability of interpretable data. In the present example, for a sample fraction containing 1.5 chromosomes from Chr 6 it is contemplated that 72% of the fractions will produce data with enough asymmetry to allow for distinguishing the M6 allele from the P6 allelic detection signal (e.g., for phasing the alleles), or vice versa. Therefore, as few as 10 fractions of 15 nl has a 99.99% probability of at least one of the fractions providing useable data (e.g., an asymmetry of underlying components greater than the resolution of the downstream processes, such as sequencing, microarray assays, PCR, etc.). However, the character of the graph, or the number of fractions for assay that will yield interpretable data, is contemplated to change dependent on the resolution of the method used for data acquisition (e.g., sequencing methods vs. microarray methods vs. PCR methods, etc.).

Once fractioned, the sample is processed according to the needs of the investigator (e.g., sequencing, microarray analysis, epigenetic analysis, PCR, etc.). As previously described, the outcome of the asymmetric distribution method provides numerous sample aliquots or fractions, all of which can be analyzed directly by an investigator or stored appropriately for later analysis. An investigator may only wish to analyze the sample aliquots which contain the highest degree of asymmetry thereby providing the highest signal-to-noise ratio, at which point the investigator can characterize the fractions for that characteristic and use only those that fit the needs of the investigator in that regard. Exemplary downstream applications which can be used to analyze asymmetrically distributed sample fractions include, but are not limited to, DNA library preparation, amplification (e.g., PCR, qPCR, MDA, and the like), microarray analysis, sequencing, genotyping and haplotyping, as previously discussed.

### Example 2

### Determination of Haplotype Using Asymmetric Sample Distribution Method

Human genomic DNA from a normal individual was diluted to 0.5 haploid copies per 3 μl water (5.00E-07 μg/μl). Diluted genomic DNA (3 μl) was aliquoted into multiple tubes resulting in, on average, 0.5 haploid copies of the human genome in each tube. To each tube, 3 μl of buffer D2 (2.75 μl DLB buffer with 0.25 μl 1M DTT) was added (Qiagen REPLI-g® UltraFast Mini Handbook, Catalog #150035) followed by a 10 minute incubation at 4° C. in a BioRad DNA Engine thermal cycler (BioRad Part # PTC-0200G). 3 μl of REPLI-g UltraFast Stop Solution was added followed by the addition of 33 μl of Mastermix. The Mastermix contained 30 μl REPLI-g UltraFast Reaction Buffer, 2 μl REPLI-g UltraFast DNA Polymerase and 1 μl of 7.56 mM humanized 9-mer pool containing 6,000 oligonucleotides (for a final concentration of 0.03 μM per oligo). The reactions were incubated for 90 minutes at 30° C. in a BioRad Tetrad2 thermal cycler (BioRad Part # PTC-0240G) followed by heat-inactivation of the REPLI-g UltraFast DNA Polymerase by heating the sample for 3 min at 65° C.

The Multiple Displacement Amplification (MDA) products were purified using DNA Clean & Concentrator™-5 spin columns (Zymo Research Catalog # D4003) according to the manufacturer's protocol. A DNA binding Buffer to MDA product volume ratio of 2:1 was used. Purified MDA product was eluted in 12 μl water.

Infinium® genotyping assays using Illumina® 300K HumanCytoSNP-12 BeadChips according to instructions in Illumina protocol 11230143 Rev A were run using 4 μl of each purified product. After scanning the BeadChips on an iScan with iCS 3.3.28 (Infinium II Assay Lab Setup and Procedures Guide, Illumina part #11207963) data was imported into the GenomeStudio™ 2008.1 Framework using the GenomeStudio™ Genotyping Module v1.0 (Illumina Part #11318815).

Scatterplots of the raw X and Y intensities per sample were used to indicate the presence of hemizygotes of two loci, arbitrarily called A and B, in the starting material, for example (X,0) and (0,Y) whereas an A/A or A/0 genotype would result in datapoints along the X axis, a B/B or B/0 genotype would result in datapoints along the Y axis and an A/B genotype would result in datapoints along the diagonal between the X and Y axis. Combined whole genome (X,Y) datapoints would indicate a heterozygous allele which would be present if greater than one haploid copy of the human genome was present in the starting material.

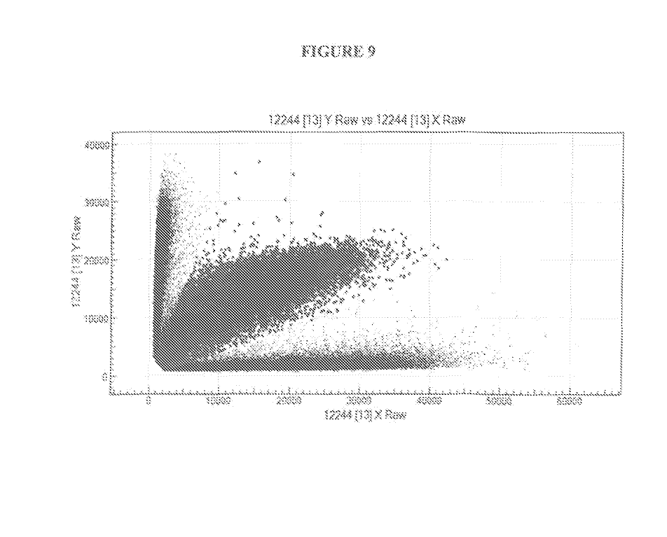

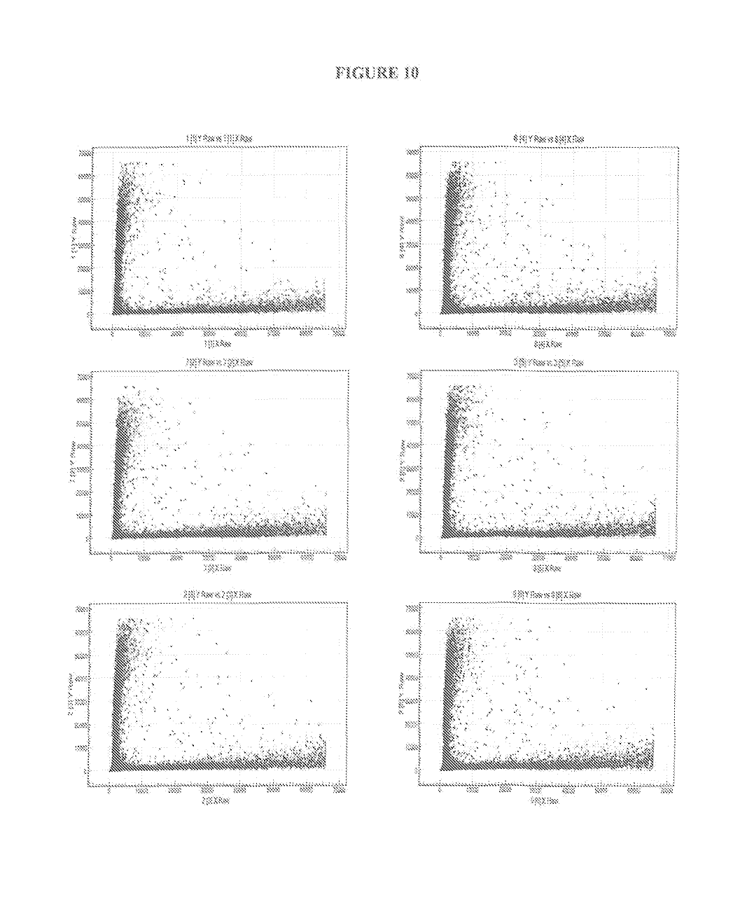

FIGS. 9-10 demonstrate the ability of the method to resolve heterozygous SNPs into their haploid components. FIG. 9 scatterplot represents exemplary raw intensities of a normal diploid individual demonstrating B/B genotype loci intensities concentrated along the Y axis (0,Y), A/A genotype loci intensities concentrated along the X axis (X,0), and A/B genotype loci intensities concentrated midway between the X and Y axis (X/Y). FIG. 10 represents exemplary raw intensities of 6 of the 12 diluted samples for the A & B loci in the diploid sample of FIG. 9. The A/A loci intensity data are concentrated along the X axis, whereas the B/B loci intensity data are concentrated along the Y axis.

### Example 3

### Determination of the X-Chromosomal Duchenne Muscular Dystrophy Gene (DMD) Haplotypes in a Mixture of Two Male Genomic DNAs Using Asymmetric Sample Distribution Method and Next Generation Sequencing

Two normal male genomic DNA samples with sequenced genomes, NA18507 (Bentley et al., 2008, Nature 456: 53-59) and HG01377 (Durbin et al., 2010, Nature 467: 1061-1073) (Coriell Cell Repositories, Camden, N.J.), were combined at equal ratios yielding an artificial sample with diploid X-chromosomes with known haplotypes. The sample was diluted and distributed into 96 aliquots at 0.2 haploid copies per aliquot (6.00E-07 μg/ul). Each aliquot of diluted template DNA was individually amplified with MDA as described in Example 2. In order to assess hemizygosity and genome coverage of the aliquots, 4 μl of the amplified material was assayed by Infinium® genotyping on an Illumina® 300K HumanCytoSNP-12 BeadChip. One hundred nanograms of purified MDA product or 50 ng of undiluted genomic DNA was converted to sequencing libraries using Nextera™ technology according to the manufacturer's protocol (Illumina, Inc., San Diego, Calif.). Each sample was barcoded during the limited cycle PCR.

Up to 12 sequencing libraries were pooled prior to sequencing for a total of eight pools. Sequencing libraries were purified with AMPure XP beads at a 0.6 ratio according to the manufacturer's guidelines (Beckman Coulter Genomics, Danvers, Mass.). A probe pool was designed for the targeted pull-down of a 1 Mb contiguous region of the DMD gene. The biotinylated probes were 80 nt long and designed to hybridize to the 5′ region of the DMD gene at 190-370 bp intervals. After pooling, the sequencing libraries were enriched for the 1 Mb DMD gene region following the protocol of the TruSeq™ Custom Enrichment Kit (Illumina, San Diego, Calif.). Enriched, indexed libraries were sequenced on a Genome Analyzer IIx (Illumina, San Diego, Calif.) using paired end sequencing for 75+35 or 75+75 read lengths. Each lane contained one pool of 12 samples. Each male was separately enriched and sequenced to confirm the true haplotype structure within the mixed DNA and the mixed sample was independently enriched and sequenced to ascertain all of the heterozygous SNPs within the region and to assess performance of the DMD oligo enrichment pool.

The sequence reads were demultiplexed and aligned to the human genome using the Illumina CASAVA v1.8.1 software package, creating aligned bam files for each indexed dilution sample. Contiguous regions were extracted from the bam files with SAMtools (Li et al., 2009, Bioinformatics 25: 2078-2079) and target cut (Kitzman et al., 2011, Nat. Biotechnol 29: 59-63). Within each contiguous fragment, base calls were made at the positions of known SNPs from the “diploid” sequencing data. Fragments were broken into continuous homozygous segments, i.e. overlapping DNA fragments were removed and ReFHap (Duitama et al., 2011, Nucleic Acids Res. doi:10.1093/nar/gkr1042) was run to merge haplotyped fragments into haplotype blocks. The resulting haplotypes were compared with the known haplotypes of the two individual male gDNAs.

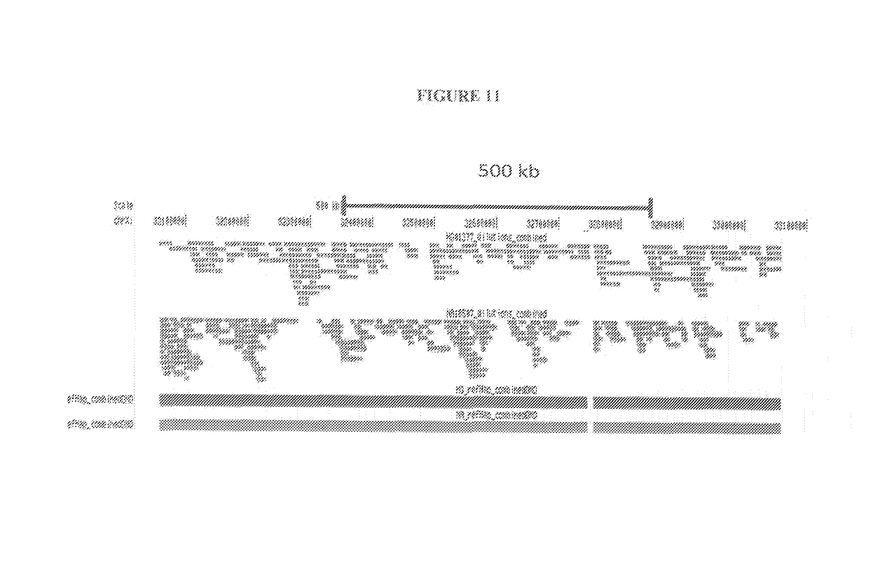

FIG. 11 shows the individual continuous homozygous aligned segments derived from HG01377 (top) and NA18507 (bottom) in the top panel and the merged haplotype blocks in the bottom panel as custom tracks loaded into the University of California Santa Cruz Genome Browser. The gap between the two merged haplotype blocks is due to an unalignable region in the human genome. The total haplotyped region is 989 kb and the mean haplotype block size is 494 kb.

### Example 4

### Haplotyping of a Whole Human Genome Using Asymmetric Sample Distribution Method and Next Generation Sequencing

Human genomic DNA from a normal individual, NA18506 (Coriell Cell Repositories, Camden, New Jersey), was diluted to 0.5 or 1.0 haploid copies per 1 μl water (1.50E-06 μg/μl or 3.00E-06 μg/μl). Diluted genomic DNA (1 μl) was aliquoted into 24 tubes per dilution resulting in, on average, 0.5 or 1.0 haploid copies of the human genome in each tube. To each tube, 1 μl of buffer D1 (0.125 μl DLB buffer with 0.875 μl water) was added (Qiagen REPLI-g® UltraFast Mini Handbook, Catalog #150035) followed by a 3 minute incubation at room temperature. One microliter of buffer N1 (0.2 μl REPLI-g UltraFast Stop Solution with 1.8 μl water) was added followed by the addition of 17 μl of Mastermix. The Mastermix contained 15 μl REPLI-g UltraFast Reaction Buffer, 1 μl REPLI-g UltraFast DNA Polymerase and 1 μl of water. The reactions were incubated for 90 minutes at 30° C. in a BioRad Tetrad2 thermal cycler (BioRad Part # PTC-0240G) followed by heat-inactivation of the REPLI-g UltraFast DNA Polymerase by heating the sample for 3 min at 65° C.

The MDA products were purified using DNA Clean & Concentrator™-5 spin columns (Zymo Research Catalog # D4003) according to the manufacturer's protocol. A DNA binding Buffer to MDA product volume ratio of 2:1 was used. Purified MDA product was eluted in 17 μl water. Purified MDA product (15 μl) was converted to sequencing libraries using Nextera™ technology according to the manufacturer's protocol (Illumina, Inc., San Diego, Calif.) with the exception that the Nextera enzyme was diluted 100 fold to compensate for the low DNA template input quantities into the tagmentation reaction and to increase the ratio of dsDNA/Nextera enzyme. This prevents the generation of library insert sizes that are too small. Each sample was barcoded during the limited cycle PCR. Up to 12 sequencing libraries were pooled prior to sequencing for a total of four pools. Sequencing libraries were purified with AMPure XP beads at a 0.6 ratio according to the manufacturer's guidelines (Beckman Coulter Genomics, Danvers, Mass.). The libraries were sequenced on a HiSeq 2000 Sequencing System (Illumina, San Diego, Calif.) using paired end sequencing for 100+100 read lengths. Each pool of 12 samples was sequenced in 2 lanes. Analysis of the sequence reads was done as described in Example 4. Accuracy was verified by comparison to resolved haplotypes obtained through statistical computation from the parental genotypes.

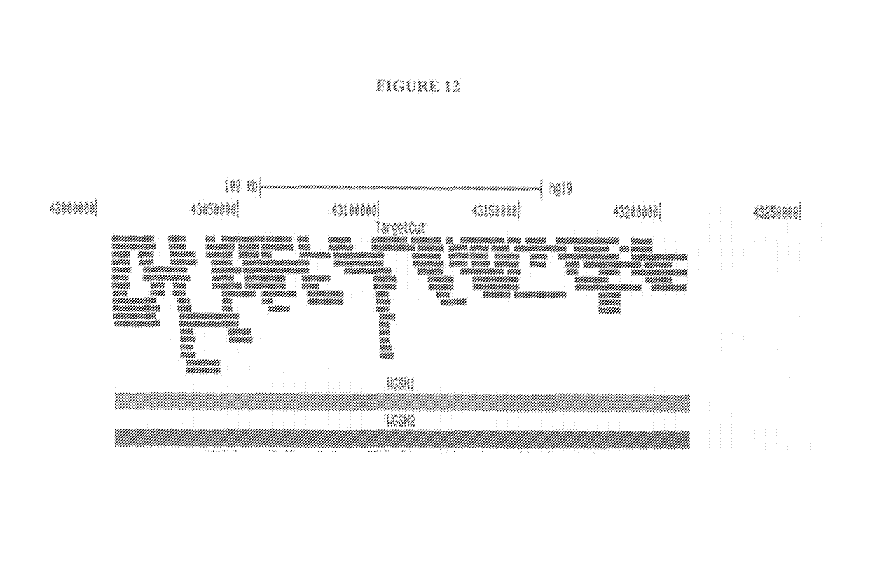

FIG. 12 shows an example of individual continuous homozygous segments in the top panel and the merged haplotype blocks in the bottom panel as custom tracks loaded into the University of California Santa Cruz Genome Browser. This example demonstrates that haplotyping can be performed on a whole genome diploid sample. The largest accurate haplotype block obtained was 303.5 kb and a total of 1.27 Gb was haplotype-resolved.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

1. A method for determining the haplotype of a nucleic acid sample, comprising providing one or more fractions of a nucleic acid sample wherein the maternal and paternal chromosomal contribution is unequal, detecting an imbalance between two or more sequences of interest in the one or more fractions of a nucleic acid sample and determining a haplotype of the nucleic acid sample based on said detectable imbalance.

2. The method of claim 1, wherein said nucleic acid sample is from a genome or fragments thereof.

3. The method of claim 2, wherein said genome is from one or more cells.

4. The method of claim 3, wherein said one or more cells are approximately 10-100 cells.

5. The method of claim 1, wherein said nucleic acid sample is from a mammal.

6. The method of claim 5, wherein said mammal is a human.

7. The method of claim 1, wherein the maternal and paternal chromosomes comprise one or more variant sequences selected from a group consisting of single nucleotide polymorphisms, copy number variants, genomic insertions and genomic deletions.

8. The method of claim 1, wherein said unequal contribution of maternal and paternal chromosomes comprises a ratio of chromosomes other than a 1:1 ratio.

9. The method of claim 1, wherein said haplotype is determined by fluorescence.

10. The method of claim 1, wherein said haplotype is determined by a nucleic acid sequencing technique.

11. The method of claim 1, wherein said haplotype is determined by a genotyping technique carried out on a microarray.

12. The method of claim 1, wherein said haplotype is determined by quantitative polymerase chain reaction.

13. A method for preparing a fraction for haplotype determination comprising:
a) providing a nucleic acid sample comprising a ratio of maternal and paternal chromosomal components that is natural to the sample, and
b) generating a plurality of fractions wherein one or more fractions comprises a skewed ratio of maternal and paternal chromosomal components, wherein the skewed ratio is substantially different from the ratio that is natural to the individual, thereby preparing a fraction for haplotype determination.

14. The method of claim 13, wherein said generating comprises asymmetrically distributing maternal and paternal chromosomal components to one or more fractions of a plurality of fractions.

15. The method of claim 13, wherein said generating comprises differentially degrading one or more of the maternal or paternal chromosomal components in one or more fractions of the plurality of fractions.

16. The method of claim 13, wherein said generating comprises differentially amplifying one of the maternal or paternal chromosomal components in one or more fractions of the plurality of fractions.

17. The method of claim 13, wherein said nucleic acid sample is from a mammal.

18. The method of claim 17, wherein a mammal is a human.

19. The method of claim 13, wherein said nucleic acid sample is from a plurality of cells.

20. The method of claim 19, wherein said plurality of cells is metaphase synchronized.

21. The method of claim 19, wherein said plurality of cells is approximately 5 to approximately 300 cells.

22. The method of claim 19, wherein said plurality of cells is approximately 10 to approximately 100 cells.

23. A method for determining the haplotype for a plurality of sequences of interest in a sample comprising:
a) providing one or more fractions from claim 13,
b) creating a library from said one or more fractions,
c) detecting a detectable signal for the plurality of sequences of interest,
d) determining the haplotype for the plurality of sequences of interest based on said differences in the detectable signals.

24. The method of claim 23, wherein the two or more sequences of interest are on the same chromosome.

25. The method of claim 23, wherein the two or more sequences of interest are located at two or more different loci on the same chromosome.

26. The method of claim 24, wherein the two or more different loci on the same chromosome are separated by at least 10000 nucleotides.

27. The method of claim 24, wherein the two or more different loci are located on the same chromosome and are separated by at least 100000 nucleotides.

28. The method of claim 24, wherein the two or more different loci are located on the same chromosome and are separated by at least 100000000 nucleotides.

29. The method of claim 24, wherein the two or more different loci are located on the same chromosome and are separated by at least 200000000 nucleotides.

30. The method of claim 23, wherein said one or more fractions are from an individual organism.

31. The method of claim 23, wherein said one or more fractions are from a mammal.

32. The method of claim 23, wherein said one or more fractions are from a human.

33. The method of claim 23, further comprising prior to step b) determining the ratio of maternal and paternal chromosomal.

34. The method of claim 23, wherein said determining the haplotype comprises quantitative polymerase chain reaction analysis of a fraction.

35. The method of claim 23, wherein said determining the haplotype comprises microarray analysis of a fraction.

36. The method of claim 23, wherein said determining the haplotype comprises detecting differences in the number of sequence reads for each of the plurality of sequences of interest, matching the sequences of interest that have similar sequence reads, and determining the haplotype based on the matched sequences of interest.

37. The method of claim 23, wherein said detectable signal is fluorescence.

38. The method of claim 36, wherein said detectable signal is fluorescence.

39. The method of claim 23, wherein the two or more sequences of interest are selected from the group comprising alleles, single nucleotide polymorphisms, copy number variants, genomic insertions and genomic deletions.

40. The method of claim 23, wherein said detecting comprises a nucleic acid sequencing technique.

41. The method of claim 23, wherein said detecting comprises a genotyping technique carried out on a microarray.

42. The method of claim 23, wherein said detecting comprises a quantitative polymerase chain reaction genotyping technique

43. The method of claim 40, wherein the sequencing technique detects differences in the number of reads at the plurality of sequences of interest out of a total number of reads at the plurality of sequences of interest.

44. The method of claim 43, wherein detecting a number of reads comprises detecting the number of fluorescent signals generated at the plurality of sequences of interest.

45. A method of determining the phase of alleles at a plurality of loci, comprising:
a) providing an asymmetric distribution of nucleic acid molecules wherein the asymmetric distribution comprises a plurality of fractions, wherein the individual fractions comprise multiple copies of the alleles, and wherein the individual fractions comprise different quantities of the alleles,
b) distinguishing the alleles in the copies of the nucleic acid molecules that are present in one or more individual fractions;
c) evaluating the different quantities of the alleles that are present in the one or more individual fractions; and
d) determining the phase of the alleles at a plurality of loci from the distinguishing of the alleles and from the evaluating the different quantities of the alleles.

46. The method of claim 45, wherein said evaluating comprises detecting differences in the number of fluorescent sequencing reads of the alleles at a plurality of loci out of a total number of reads.

47. The method of claim 45, wherein the nucleic acid molecules are from an individual organism.

48. The method of claim 45, wherein the evaluating of the different quantities comprises determining a ratio of alleles at the plurality of loci.

49. The method of claim 45, wherein the distinguishing of the alleles comprises determining the identity of one or more nucleotides present at the plurality of loci.

50. The method of claim 45, wherein the distinguishing of the alleles comprises a nucleic acid sequencing technique.

51. The method of claim 45, wherein the distinguishing of the alleles comprises a genotyping technique carried out on a microarray.

52. The method of claim 45, wherein the plurality of loci are located on the same chromosome and are separated by at least 10000 nucleotides.

53. The method of claim 45, wherein the plurality of loci are located on the same chromosome and are separated by at least 100000 nucleotides.

54. The method of claim 45, wherein the plurality of loci are located on the same chromosome and are separated by at least 100000000 nucleotides.

55. The method of claim 45, wherein the plurality of loci are located on the same chromosome and are separated by at least 200000000 nucleotides.

56. A nucleic acid fraction for determining a haplotype wherein said nucleic acid fraction comprises asymmetrically distributed maternal and paternal chromosomal components wherein said asymmetrically distributed chromosomal components is a skewed ratio of maternal to paternal chromosomal components that is different from the ratio that is natural to the individual.
